(19) United States
(12) Patent Application Publication
(10) Pub. No.: US 2024/0366536 A1
(43) Pub. Date: Nov. 7, 2024

(54) MODULATORS OF A POTASSIUM CHANNEL AND OF TRPV1 CHANNEL AND USES THEREOF

(71) Applicant: Bsense Bio Therapeutics Ltd., Ness Ziona (IL)

(72) Inventors: Adi RAVEH, Rehovot (IL); Alon SILBERMAN, Givatayim (IL); Haim BELINSON, Even Yehuda (IL); Alan Daniel BROWN, Deal (GB)

(73) Assignee: Bsense Bio Therapeutics Ltd., Ness Ziona (IL)

(21) Appl. No.: 18/776,348

(22) Filed: Jul. 18, 2024

Related U.S. Application Data
(63) Continuation of application No. PCT/IL2023/050058, filed on Jan. 18, 2023.
(60) Provisional application No. 63/300,293, filed on Jan. 18, 2022.

(51) Int. Cl. A61K 31/165; A61K 31/404; A61K 31/4439; A61P 25/02; A61P 25/04; C07C 233/22; C07C 233/23; C07C 321/30; C07D 209/08; C07D 209/18; C07D 401/04; C07D 403/12; C07D 405/12; C07D 405/14

(52) U.S. Cl. CPC A61K 31/165; A61K 31/404; A61K 31/4439; A61P 25/02; A61P 25/04; C07C 233/22; C07C 233/23; C07C 321/30; C07D 209/08; C07D 209/18; C07D 401/04; C07D 403/12; C07D 405/12; C07D 405/14

(57) ABSTRACT

Compounds represented by Formula I:

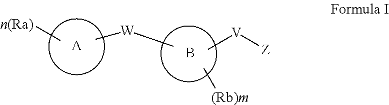

wherein A, B, W, V, Z, Ra, Rb, n and m are as defined in the instant specification, or by Formula Ia, Ib, III, IV or V, as defined in the instant specification, and uses thereof in modulating an activity of a voltage-dependent potassium channel and/or a TRPV1 channel and in treating a medical condition associated with an activity of these channels, such as medical conditions associated with neuronal hyper-excitability, are provided.

### RELATED APPLICATIONS

This application is a Continuation of PCT Patent Application No. PCT/IL2023/050058 having International filing date of Jan. 18, 2023, which claims the benefit of priority under 35 USC § 119(e) of U.S. Provisional Patent Application No. 63/300,293 filed on Jan. 18, 2022. The contents of the above applications are all incorporated by reference as if fully set forth herein in their entirety.

### FIELD AND BACKGROUND OF THE INVENTION

The present invention, in some embodiments thereof, relates to novel analogs of diphenylamine and, more particularly, but not exclusively, to newly designed diphenylamine analogs featuring a dual activity as modulators of both Kv7.2/3 and TRPV1 channels, which are usable in the treatment of medical conditions that are related to these channels, including, but not limited to, medical conditions associated with neuronal hyper-excitability such as pain, tinnitus and pruritus.

Voltage-dependent potassium (Kv) channels conduct potassium ions (K⁺) across cell membranes in response to change in the membrane voltage and thereby can regulate cellular excitability by modulating (increasing or decreasing) the electrical activity of the cell.

Functional Kv channels exist as multimeric structures formed by the association of either identical or dissimilar Kv alpha and/or Kv beta subunits. The alpha subunits comprise six transmembrane domains, a pore-forming loop and a voltage-sensor and are arranged symmetrically around a central pore. The beta or auxiliary subunits interact with the alpha subunits and can modify the properties of the channel complex to include, but not be limited to, alterations in the channel's electrophysiological or biophysical properties, expression levels or expression patterns.

Nine Kv channel alpha subunit families have been identified and are termed Kv1-Kv9. As such, there is an enormous diversity in Kv channel function that arises as a consequence of the multiplicity of sub-families, the formation of both homomeric and heteromeric subunits within sub-families and the additional effects of association with beta subunits [M. J. Christie, Clinical and Experimental Pharmacology and Physiology, 1995, 22 (12), 944-951].

The Kv7 channel family consists of at least five members which include one or more of the following mammalian channels: Kv7.1, Kv7.2, Kv7.3, Kv7.4, Kv7.5 and any mammalian or non-mammalian equivalent or variant (including splice variants) thereof. Alternatively, the members of this family are termed KCNQ1, KCNQ2, KCNQ3, KCNQ4 and KCNQ5, respectively [Dalby-Brown et al., Current Topics in Medicinal Chemistry, 2006, 6, 999-1023].

The five members of this family differ in their expression patterns. The expression of Kv7.1 is restricted to the heart, peripheral epithelial and smooth muscle, whereas the expression of Kv7.2-Kv7.4 is limited to the nervous system to include the hippocampus, cortical neurons and dorsal root ganglion neurons [for a review see, for example, Delmas P. & Brown D., Nature, 2005, 6, 850-862].

The neuronal Kv7 channels have been demonstrated to play key roles in controlling neuronal excitation. Kv7 channels, in particular Kv7.2/Kv7.3 heterodimers, underlie the M-current, a non-activating potassium current found in a number of neuronal cell types. The current has a characteristic time and voltage dependence that results in stabilization of the membrane potential in response to multiple excitatory stimuli. In this way, the M-current is central to controlling neuronal excitability [for a review, see, for example, Delmas. P & Brown. D, Nature, 2005, 6, 850-862].

Potassium channels have been associated with a number of physiological processes, including regulation of heartbeat, dilation of arteries, release of insulin, excitability of nerve cells, and regulation of renal electrolyte transport. Modulators of potassium channels are therefore prime pharmaceutical candidates, and the development of new modulators as therapeutic agents is an on-going research effort.

Thus, given the key physiological role of Kv7 channels in the nervous system and the involvement of these channels in a number of diseases, the development of modulators of Kv7 channels is highly desirable.

Potassium channels modulators are divided to channel-openers and channel-blockers. A potassium channel opener that has gained much attention is retigabine (N-(2-amino-4-(4-fluorobenzylamino)-phenyl)carbamic acid ethyl ester). Retigabine is highly selective for KCNQ2-5-type potassium channels. Use of retigabine for treating neuropathic pain was disclosed in, for example, U.S. Pat. No. 6,117,900 and EP 1223927. Compounds related to retigabine have also been proposed for use as potassium channel modulators (see, for example, U.S. Pat. No. 6,472,165).

However, retigabine has been reported to have multiple effects in neuronal cells. These include sodium and calcium channel blocking activity (Rundfeldt, C, 1995, Naunyn-Schmiederberg's Arch Pharmacol, 351 (Suppl): R160) and effects on GABA (7-aminobutyric acid) synthesis and transmission in rat neurons (Kapetanovic, I. M., 1995, Epilepsy Research, 22, 167-173, Rundfeldt, C, 1995, Naunyn-Schmiederberg's Arch Pharmacol, 351 (Suppl):R160).

Other KCNQ potassium channel modulators have been described in, for example, U.S. patent application Ser. No. 10/075,521, which teaches 2,4-disubstituted pyrimidine-5-carboxamide derivatives as Kv7 modulator; U.S. patent application Ser. No. 10/160,582, which teaches cinnamide derivatives as voltage-dependent potassium channel modulators; U.S. Pat. No. 5,565,483 and U.S. patent application Ser. Nos. 10/312,123, 10/075,703 and 10/075,522, which teach 3-substituted oxindole derivatives as voltage-dependent potassium channel modulators; U.S. Pat. No. 5,384,330, which teaches 1,2,4-triamino-benzene derivatives as potassium channel modulators; and U.S. Pat. No. 6,593,349 which teaches bisarylamines derivatives as voltage-dependent potassium channel modulators. U.S. Pat. No. 6,291,442 teaches compounds comprising two or three aromatic rings having a free carboxyl or a carboxyl being linked, via an ester bond, to a lower alkyl ester, attached to one of the rings, for the modulation of Shaker class of voltage gated potassium channels.

WO 2004/035037 and U.S. Patent Application Publication No. 20050250833 teach derivatives of N-phenylanthranilic acid and of 2-benzimidazolone as potassium channel openers, especially openers of voltage-dependent potassium channels such as Kv7.2, Kv7.3 and Kv7.2/7.3 channels, as well as neuron activity modulators.

WO 2009/037707 teaches additional derivatives of N-phenylanthranilic as potassium channel and/or TRPV1 modulators. An exemplary modulator disclosed in WO 2009/037707 is referred to as NH29:

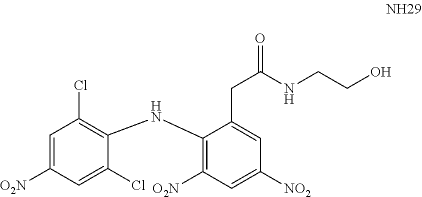 [SMILES: O=C(Cc1cc([N+](=O)[O-])cc([N+](=O)[O-])c1Nc1c(Cl)cc([N+](=O)[O-])cc1Cl)NCCO]

WO 2009/071947 and WO 2010/010380 teach derivatives of diphenylamine as potassium channel modulators. Exemplary modulators disclosed in these patent applications are referred to as NH34 and NH43:

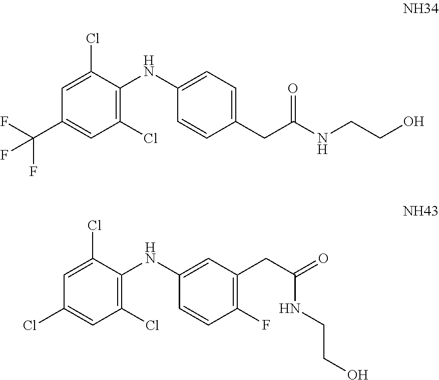 [SMILES: O=C(Cc1ccc(Nc2c(Cl)cc(C(F)(F)F)cc2Cl)cc1)NCCO ; O=C(Cc1cc(Nc2c(Cl)cc(Cl)cc2Cl)ccc1F)NCCO]

Transient receptor potential vanilloid type 1 (TRPV1) receptor is a ligand-gated non-selective cation channel activated by heat (typically above 43° C.), low pH (<6) and endogenous lipid molecules such as anandamide, N-arachidonoyl-dopamine, N-acyl-dopamines and products of lipoxygenases (e.g., 12- and 15-(S)-HPETE) termed endovanilloids. Apart from peripheral primary afferent neurons and dorsal root ganglia, TRPV1 receptor is expressed throughout the brain. Recent evidence shows that TRPV1 receptor stimulation by endocannabinoids or by capsaicin leads to analgesia and this effect is associated with glutamate increase and the activation of OFF cell population in the rostral ventromedial medulla (RVM).

TRPV1 has also been found to be involved in the regulation of body temperature, anxiety and mediation of long-term depression (LTD) in the hippocampus. TRPV1 channels are also located on sensory afferents, which innervate the bladder. Inhibition of TRPV1 has been shown to ameliorate urinary incontinence symptoms.

TRPV1 modulators have been described in, for example, WO 2007/054480, which teaches the effect of 2-(benzimidazol-1-yl)-acetamide derivatives in the treatment of TRPV1 related diseases. WO 2008/079683 teaches compounds being a conjugated two-ring system of cyclohexyl and phenyl for inhibiting TRPV1 receptor. EP 01939173 teaches O-substituted-dibenzyl urea- or thiourea-derivatives as TRPV1 receptor antagonists. WO 2008/076752 teaches benzimidazole compounds as potent TRPV1 modulators and EP 01908753 teaches TRPV1 modulators being heterocyclidene acetamide derivatives.

The potassium channel Kv7.2/3 and the cation non-selective channel TRPV1 are uniquely co-expressed in afferent peripheral sensory neurons (DRG sensory neurons), which convey sensory signals and have opposite functions. TRPV1 channels trigger the pain signals, while Kv7.2/3 channels inhibit them. Compounds that simultaneously function as activators (e.g., openers) of Kv7.2 and inhibitors (e.g., blockers) of TRPV1 can depress neuronal hyper-excitability associated with medical conditions such as pain, tinnitus and pruritus.

WO 2019/073471 discloses various modifications performed to the structures taught in WO 2009/071947 and WO 2010/010380, which were found to exhibit dual modulation of both a voltage-dependent potassium channel and of TRPV1. Two of the potential candidates disclosed in WO 2019/073471 are referred to therein as NH91 and NH101:

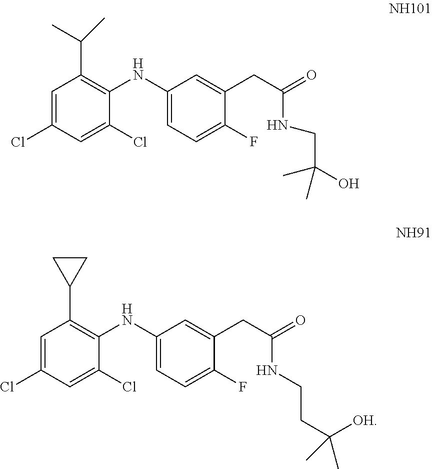 [SMILES: CC(C)c1cc(Cl)cc(Cl)c1Nc1ccc(F)c(CC(=O)NCC(C)(C)O)c1 ; CC(C)(O)CCNC(=O)Cc1cc(Nc2c(Cl)cc(Cl)cc2C2CC2)ccc1F]

### SUMMARY OF THE INVENTION

According to an aspect of some embodiments of the present invention there is provided a compound represented by Formula I:

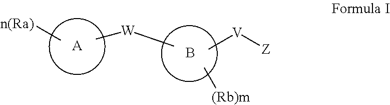

A and B are each independently selected from an aryl and a heteroaryl;
W is selected from —S—, —O—, —CR₁(OH)—, —C(═O), and NRx, wherein Rx forms a nitrogen-containing heterocyclic ring with one of the Ra substituents of ring A;
n is an integer of from 1 to 5;
m is an integer of from 0 to 4;
Ra and Rb are each independently a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Ra substituents form together an alicyclic or heterocyclic ring, at least one of the Ra substituents forms together with Rx, if present, the nitrogen-containing heterocyclic ring and/or at least two of the Rb substituents form together an alicyclic or heterocyclic ring, wherein when n is greater than 1, each Ra is the same or different substituent, and when m is greater than 1, each Rb is the same or different substituent;
R₁, if present, is hydrogen or alkyl;
V is —(CR₂R₃)k-U;
k is 0, 1 or 2;
R₂ and R₃ are each independently hydrogen, alkyl, cycloalkyl and aryl;
U is amide (—C(═O)—NR₁₀—) or an isostere thereof; and
Z is represented by Formula II:

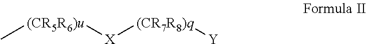

wherein:
u and q are each independently an integer of from 0 to 4, provided that u+q is at least 2;
X is selected from —O— and —NR₉—, or is absent;
Y is a polar hydrophilic group such as OR₁₁, SR₁₁, amine (NR₁₂R₁₃), or amide (—NR₁₂—C(═O)—R₁₄;
R₅, R₆, R₇ and R₈ are each independently selected from hydrogen, halo, alkyl, haloalkyl, cycloalkyl, heteroalicyclic, aryl, alkylamine, alkoxy, haloalkoxy, hydroxy, ether and aryloxy, or, alternatively, two of R₅, R₆, R₇, R₈ and R₉ form together an alicyclic or heteroalicyclic ring; and R₉, R₁₀, R₁₁, R₁₂, R₁₃ and R₁₄ are each independently selected from hydrogen, alkyl, cycloalkyl, and aryl, or, alternatively, two of R₅, R₆, R₇, R₈, R₉ and R₁₁ or two of R₅, R₆, R₇, R₈, R₉, R₁₂ and R₁₃ form together an alicyclic or heteroalicyclic ring.

According to some of any of the embodiments described herein, A and B are each an aryl, for example, A and B are each phenyl.

According to some of any of the embodiments described herein, the compound is represented by Formula Ia:

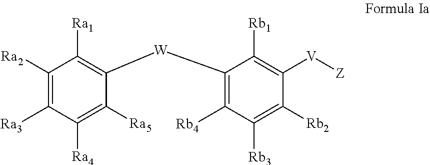

wherein:
W, V and Z are as defined for Formula I;
Ra1-Ra5 are each independently as defined herein for Ra; and
Rb1-Rb4 are each independently as defined herein for Rb.

According to some of any of the embodiments described herein, m is other than 0 and at least one of the Rb substituent(s) is halo.

According to some of any of the embodiments described herein, m is 1.

According to some of any of the embodiments described herein, the halo is at the para position with respect to the W.

According to some of any of the embodiments described herein, Rb2 is halo, for example, fluoro.

According to some of any of the embodiments described herein, B is a heteroaryl, for example, a nitrogen-containing heteroaryl such as pyridine, pyrazole, pyrrole, imidazole, and the like. According to some embodiments, B is pyridine.

According to some of any of the embodiments described herein, A is aryl, for example phenyl.

According to some of any of the embodiments described herein, the compound is represented by Formula Ib:

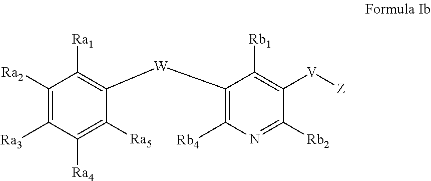

wherein:
W, V and Z are as defined for Formula I;
Ra1-Ra5 are each independently as defined herein for Ra; and
Rb1, Rb2 and Rb4 are each independently as defined herein for Rb.

According to some of any of the embodiments described herein, n is 3, 4 or 5, and in some embodiments, n is 3.

According to some of any of the embodiments described herein, at least two of the Ra substituents are selected from halo and alkoxy.

According to some of any of the embodiments described herein, at least two of the Ra substituents are each independently halo, for example, chloro.

According to some of any of the embodiments described herein, at least one of the Ra substituent(s) is selected from alkyl, haloalkyl, cycloalkyl, aryl, alkoxy, alkylamine, heteroaryl and heteroalicyclic, or, alternatively, two Ra substituents form together a cyclic ring.

According to some of any of the embodiments described herein, at least one of the Ra substituent(s) is alkyl or cycloalkyl.

According to some of any of the embodiments described herein, at least one of the Ra substituents is at the ortho position with respect to the W.

According to some of any of the embodiments described herein, at least one of the Ra substituent(s) is alkyl, haloalkyl, cycloalkyl, aryl, alkoxy, alkylamine, heteroaryl or heteroalicyclic, and is at the ortho position with respect to the W.

According to some of any of the embodiments described herein, n is 3, one of the Ra substituent(s) is alkyl, haloalkyl, cycloalkyl, aryl, alkoxy, alkylamine, heteroaryl or heteroalicyclic, and is at the ortho position with respect to the W, and the two other Ra substituents are each halo.

According to some of any of the embodiments described herein, n is 3;
one of the Ra substituent(s) is alkyl, haloalkyl, cycloalkyl, aryl, alkoxy, alkylamine, heteroaryl or heteroalicyclic, and is at the ortho position with respect to the W; the two other Ra substituents are each halo; m is 1; and Rb is halo and is at the para position with respect to the W.

According to some of any of the embodiments described herein, Ra1 is alkyl, haloalkyl, cycloalkyl, aryl, alkoxy, alkylamine, heteroaryl or heteroalicyclic.

According to some of any of the embodiments described herein, Ra3 and Ra5 are each independently a halo (e.g., chloro).

According to some of any of the embodiments described herein, X is absent.

According to some of any of the embodiments described herein, at least one of R₅, R₆, R₇ and R₈ is independently selected from alkyl, hydroxy, alkoxy, haloalkyl, ether, and halo, and/or at least two of R₅, R₆, R₇ and R₈ form together an alicyclic or heteroalicyclic ring.

According to some of any of the embodiments described herein, at least two of R₅, R₆, R₇ and R₈ are independently selected from alkyl, haloalkyl and halo.

According to some of any of the embodiments described herein, at least two of R₅, R₆, R₇ and R₈ are each independently an alkyl.

According to some of any of the embodiments described herein, u is 1 or 2.

According to some of any of the embodiments described herein, R₅ and R₆ are each hydrogen.

According to some of any of the embodiments described herein, q is 1 and at least one or each of R₇ and R₈ is alkyl.

According to some of any of the embodiments described herein, q is 1 and at least one or each of R₇ and R₈ is hydrogen.

According to some of any of the embodiments described herein, at least one of R₅, R₆, R₇ and R₈ is independently hydroxy, hydroxyalkyl, ether, a heteroalicyclic (e.g., oxygen-containing) or a cycloalkyl.

According to some of any of the embodiments described herein, the cycloalkyl is substituted by at least one hydroxy.

According to some of any of the embodiments described herein, at least two of R₅, R₆, R₇ and R₈ form together an alicyclic ring.

According to some of any of the embodiments described herein, X is absent, u is 1 or 2, q is 1 and R₅, R₆, R₇ and R₈ form together a cyclobutane.

According to some of any of the embodiments described herein, at least two of R₅, R₆, R₇ and R₈ form together a heteroalicyclic ring, preferably an oxygen-containing 4, 5 or 6-membered heteroalicyclic.

According to some of any of the embodiments described herein, X is absent, u is 1 or 2, q is 1 and R₅, R₆, R₇ and R₈ form together a furan.

According to some of any of the embodiments described herein, Y is OR₁₁, and R₁₁ is hydrogen.

According to some of any of the embodiments described herein, Y is OR₁₁ and Ru is an alkyl, preferably substituted by at least one hydroxy, amide and/or carboxy.

According to some of any of the embodiments described herein, Y is OR₁₁ and Ru is a heteroaryl or heteroalicyclic.

According to some of any of the embodiments described herein, Y is NR₁₂R₁₃ and R₁₂ forms together with one or more of R₅, R₆, R₇ and R₈ a nitrogen-containing heteroalicyclic.

According to some of any of the embodiments described herein, R₁₃ is hydrogen, hydroxy, alkyl, hydroxyalkyl or alkoxy.

According to some of any of the embodiments described herein, Y is —NR₁₂—C(═O)—R₁₄, and R₁₂ forms together with one or more of R₅, R₆, R₇ and R₈ a nitrogen-containing heteroalicyclic.

According to some of any of the embodiments described herein, Rx forms together with one of the Ra substituents a nitrogen-containing heterocyclic ring.

According to some of any of the embodiments described herein, Rx forms the heterocyclic ring with an Ra substituent at the ortho position with respect to W.

According to some of any of the embodiments described herein, the compound is represented by Formula III:

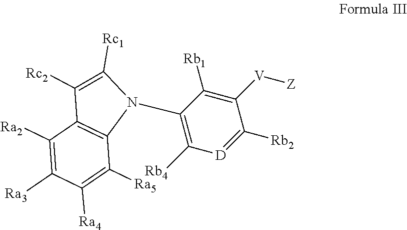

wherein:
V and Z are as defined herein for Formula I,
D is N or C—Rb3;
Ra2-Ra5, Rb1-Rb4, V and Z are as defined herein for Formula I, Ta or Tb; and
Rc1 and Rc2 are each independently a substituent as defined herein for Ra1-Ra5.

According to some of any of the embodiments described herein, Rc1 and Rc2 are each independently selected from hydrogen, alkyl, haloalkyl, and halo.

According to some of any of the embodiments described herein, W is —S—.

According to some of any of the embodiments described herein, the compound is represented by Formula IV:

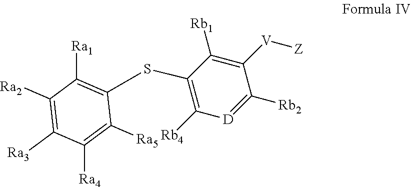

wherein:
V and Z are as defined herein for Formula I;
D is N or C—Rb3; and
Ra1-Ra5, Rb1-Rb4, V and Z are as defined herein for Formula I, Ia or Ib.

According to some of any of the embodiments described herein, Ra1 is selected from alkyl and cycloalkyl.

According to some of any of the embodiments described herein, W is —CR₁(OH).

According to some of any of the embodiments described herein, the compound is represented by Formula V:

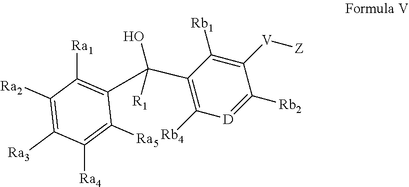

wherein:
V and Z are as defined herein for Formula I;
D is N or C—Rb3; and
Ra1-Ra5, Rb1-Rb4, R₁, V and Z are as defined herein for Formula I, Ia or Ib.

According to some of any of the embodiments described herein, Ra1 is selected from alkyl and cycloalkyl.

According to some of any of the embodiments described herein, W is —C(═O)—.

According to some of any of the embodiments described herein, the compound is represented by Formula VI:

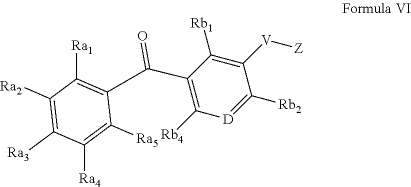

wherein:
V and Z are as defined herein for Formula I;
D is N or C—Rb3; and
Ra1-Ra5, Rb1-Rb4, V and Z are as defined herein for Formula I, Ia or Ib.

According to some of any of the embodiments described herein, Ra1 is selected from alkyl and cycloalkyl.

According to some of any of the embodiments described herein, the compound is characterized by at least one of:
LogD, determined as described herein, higher than 4, or higher than 5, or ranging between 4 and 7;
Ligand-lipophilicity efficiency (LLE), determined as described herein, higher than 3, or higher than 5;
HLM Clint, determined as described herein, lower than 100 μl/min/mg; and
Kinetic solubility higher than 20 or higher than 30 micromolar.

According to an aspect of some embodiments of the present invention there is provided a pharmaceutical composition comprising the compound as described herein in any of the respective embodiments and any combination thereof and a pharmaceutically acceptable carrier.

According to an aspect of some embodiments of the present invention there is provided a compound as described herein in any of the respective embodiments and any combination thereof or a pharmaceutical composition comprising the compound as described herein, for use in modulating an activity of a voltage-dependent potassium channel.

According to some of any of the embodiments described herein, the potassium channel is Kv7.2/7.3.

According to some of any of the embodiments described herein, the modulating comprises opening the channel.

According to an aspect of some embodiments of the present invention there is provided a compound as described herein in any of the respective embodiments and any combination thereof or a pharmaceutical composition comprising the compound as described herein, for use in modulating an activity of TRPV1.

According to some of any of the embodiments described herein, the modulating comprises inhibiting the activity of TRPV1.

According to an aspect of some embodiments of the present invention there is provided a compound as described herein in any of the respective embodiments and any combination thereof or a pharmaceutical composition comprising the compound as described herein, for use in modulating an activity of both a voltage-dependent potassium channel and TRPV1.

According to some of any of the embodiments described herein, modulating the activity of the voltage-dependent potassium channel comprises opening the channel and wherein modulating the activity of the TRPV1 channel comprises inhibiting an activity of the channel.

According to some of any of the embodiments described herein, the potassium channel is Kv7.2/7.3.

According to an aspect of some embodiments of the present invention there is provided a compound as described herein in any of the respective embodiments and any combination thereof, or a pharmaceutical composition as described herein, for use in treating a medical condition associated with an activity of a voltage-dependent potassium channel and/or a TRPV1 channel.

According to some of any of the embodiments described herein, the medical condition is neuropathic pain, pruritus and/or tinnitus.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents the chemical structures of exemplary compounds encompassed by chemotype 1 according to some embodiments of the present invention.

FIG. 2 presents the chemical structures of exemplary compounds encompassed by chemotype 2 according to some embodiments of the present invention.

FIGS. 3A-B present the chemical structures of exemplary compounds encompassed by chemotypes 3 and 4 according to some embodiments of the present invention (FIG. 3A) and the potential enzyme-catalyzed transformation therebetween (FIG. 3B).

FIGS. 4A-B present a homology model of the human TRPV1 with reference (previously described) compounds docked into the vanilloid pocket: Reference compounds NH91 (000091; bright yellow), 000228 (as disclosed, for example, in WO 2004/035037; green) and the TRPV1 established agonist resiniferatoxin (RTX) serving as a reference molecule (FIG. 4A); and a homology model of the human Kv7.2/7.3 with the previously described 000091 (NH91) docked inside the VSD (FIG. 4B). Hydrophobic gaskets and hydrophilic channels are shared structural motifs of both proteins allowing a design dual Kv7.2/7.3 and TRPV1 modulators.

FIGS. 5A-B present a representative example of TRPV1 qSAR modelling. FIG. 5A presents a superposition of exemplary potent TRPV1 inhibitors according to some embodiments of the present invention. FIG. 5B is a linear plot correlating experimental IC50 results (X axis) and the predicted qSAR IC50 values for exemplary compounds according to some embodiments of the present invention.

FIG. 6A-C present data obtained for rat DRG neuron firing in responses to current injection (FIG. 6A), Capsaicin (FIG. 6B) or both (FIG. 6C) activation with or without application of varying concentrations of AMG9810—a TRPV1 specific antagonist, compound 273—a Kv activator lacking TRPV1 inhibition, and exemplary compound 421-6—showing an exemplary Kv and TRPV1 dual targeting compound and Retigabine (RET) as an exemplary Kv7.2/3 targeting compound with similar EC50 to that of 421-6.

FIGS. 6D-E present dose response curves displaying compound 421-6 and retigabine inhibition (FIGS. 6D and 6E, respectively) of both capsaicin-induced and current-induced neuronal excitability at varying concentrations.

FIGS. 7A-C present exemplary data obtained for rat DRG neuron firing in response to current injection and Capsaicin firing response of compound 552 at 100 nM (FIG. 7A); a bar graph showing comparative rat DRG firing responses between compounds 421-6, 541, 552 and 533 (racemic), each at 100 nM; and exemplary data obtained for rat DRG neuron firing in responses to current injection and Capsaicin firing response in the presence of compound 533 (racemic mixture) at 100 nM.

FIG. 8A (Background Art) presents an experimental set up according to Zhang et al., 2005, EMBO J 24(24):4211-23 as described herein.

FIG. 8B presents representative data obtained in assays in which neurons were pretreated with NGF 100 ng/ml for 4-5 days. NGF Non-treated rat DRGs were stimulated by repeated capsaicin application with and without 421-6 at 2 μM (top panel) or 100 nM (Middle panel). Following NGF pretreatment tetanic burst of firing is observed and a reversible inhibition of 421-6 at 100 nM is shown.

FIGS. 8C-D present magnifications of the data presented in FIG. 8B (bottom panel (FIG. 8C), and data obtained following NGF pretreatment in the presence of compound 421-6 (100 nM) and compound 533 at 2 μM (FIG. 8D).

FIGS. 9A-B demonstrate the inhibition potency of compound 415 (5 μM, 1.5 minute; FIG. 9A) and of previously described compound 291 (2 μM) and compound 414 (5 μM) (FIG. 9B) on neuronal spontaneous activity in human derived sensory neurons, displaying both the high potency of the compound together with its reversible manner, with recovered firing following compound's wash away.

FIGS. 10A-D present data showing the effect of compound 533 as a racemic mixture (533R) and of its separated enantiomers, 533p1 and 533p2. FIG. 10A presents comparative plots showing hKv7.2/3 activation measured using high-content fluorescent assay, comparing hKv7.2/3 activation by 533 racemic mixture (533R) to its separated enantiomers, 533 μl and 533p2, showing lower and higher activation potencies, respectively. FIG. 10B presents comparative plots showing that each of the 533 separated enantiomers regulates differently hTRPV1, one (533 μl) inhibits hTRPV1 while the other (533p2) activates hTRPV1, with 533 racemic mixture (533R) showing an averaged response. AMG9810, a hTRPV1 antagonist served as a positive control. FIG. 10C presents the EC/IC50 values (in μM) of each of the separated enantiomers of compound 533 against the hKv7.2/3 and hTRPV1 targets (arrow denotes TRPV1 activation). FIG. 10D presents data showing that 533p2 application (100 nM) inhibits the current-evoked rat DRGs spiking, while the capsaicin-evoked rat DRGs spiking is facilitated, in agreement with 533p2 TRPV1 activation property (upper panel), whereby when 533 racemic mixture (533R) is applied, current-evoked spiking inhibition decreases, while capsaicin-induced spiking inhibition is gained.

FIGS. 11A-E present comparative plots showing the caspain-induced inhibition of hTRPV1 activity, measured using fluorescent assay, by compound 627 and AMG9810 as a positive control in hTRPV1 stably expressing cells (FIG. 11A), and in cells expressing hKv7.2/3 and hTRPV1 (FIG. 11B); comparative plots showing activation of hKv7.2/3 activity, measured using fluorescent assay, by compound 627 compared to retigabine positive control (FIG. 11C), and activation of hKv7.2/3 activity, measured using fluorescent assay, by compound 627 at very low concentrations (at the picomolar range) (FIG. 11D); and activation of hKv7.2/3 activity by compound 627 in electrophysiology, measuring whole-cell currents of hKv7.2/3 expressed in CHO cells, clamping membrane potential to −40 mV (1.5 s) (from a holding potential of −90 mV) (N=4) (FIG. 11E).

FIGS. 12A-C present whole-cell currents electrophysiology measurements (FIG. 12A) in hTRPV1 expressing CHO cells (upper panel) and in CHO cells co-expressing hKv7.2/3 and hTRPV1 upon activation (lower panel); a dose-response curve displaying the hTRPV1 inhibition-potency in CHO cells expressing hTRPV1 (right) and co-expressing with hKv7.2/3 and activated (left) (FIG. 12B); and an exemplary capsaicin-induced current in CHO cells co-expressing hTRPV1 and hKv7.2/3 without hKv7.2/3 activation, in the presence of compound 627 (FIG. 12C).

FIGS. 13A-B present exemplary capsaicin-induced current in CHO cells co-expressing hTRPV1 and hKv7.2/3 in the presence of AMG9810, a known TRPV1 inhibitor (FIG. 13A) and dose-response comparative plots displaying the hTRPV1 inhibition-potency in CHO cells expressing hTRPV1 and cells co-expressing hTRPV1 and hKv7.2/3 and activated, in the presence of compound 627, AMG9810, and AMG9810 and retigabine (RET) at 1 μM (FIG. 13B).

FIG. 14 presents rat DRG membrane-potential recording showing action-potentials trains in response to Capsaicin application in the presence of compound 627 at 1 nM (upper panel) and 0.1 nM (lower panel).

### DESCRIPTION OF SPECIFIC EMBODIMENTS OF THE INVENTION

The present invention, in some embodiments thereof, relates to novel analogs of diphenylamine and, more particularly, but not exclusively, to newly designed diphenylamine analogs featuring a dual activity as modulators of both potassium ion and TRPV1 channels, which are usable in the treatment of various pathologies that are related to these channels, such as, but not limited to, neuropathic pain.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As described in the Examples section that follows, the present inventors have conducted extensive studies aiming at uncovering analogs of diphenylamine derivatives that would exhibit improved therapeutic efficacy as dual modulators of both potassium ion (specifically Kv7.2/3) and TRPV1 channels, along with improved pharmacological profile in terms of toxicity, bioavailability, solubility and other pharmacological parameters and/or properties, compared to previously described diphenylamine derivatives.

In the course of these extensive studies, the present inventors have uncovered that certain structural modification to the structures taught in WO 2009/071947 and WO 2019/073471 lead to compounds that feature an improved performance.

Small molecules usable for the treatment of medical conditions in which dual modulation of the activity of a potassium channel and TRPV1 is desirable, have been designed and practiced. These small molecules are particularly usable in the treatment of neuropathic pain; however, these small molecules are usable also in the treatment of other medical conditions in which modulating, as described herein, an activity of one or both of potassium ion (specifically Kv7.2/3) and TRPV1 channels is beneficial.

These compounds were designed according to an approach of targeting multiple hyperexcitability related mechanisms using a single compound, to achieve greater efficacy and safety. This approach is based on the targeting of two cation channels, a ligand-gated calcium channel and a voltage-gated potassium channel, respectively, TRPV1 and Kv7.2/3, which are co-localized on sensory neurons and are recognized as prominent regulators of neuronal excitability.

The small molecules were designed upon testing the effect of variable modifications, at multiple positions, of previously uncovered small molecules, as described in Example 1. Classical and computational medicinal chemistry together with on target and off target screening methodologies were used in order to identify potent, safe and metabolically stable compounds for in-vivo testing. In addition, species selection studies were conducted so as to assist in finding the appropriate animal model.

Using classical and computational medicinal chemistry, rational structure activity relationship (SAR) studies yielded the design and planning of nearly 500 compounds, of which 326 compounds were screened for dual targeting properties. The results from these studies provided substantial understanding of the SAR relationship. The main goal was to replace the bridging aniline moiety, as it was found to exhibit a significant toxicological alert, leading to the identification of four new chemotypes with potencies in the low M range and even lower in a heterologous system of ion-channel expression in CHO cells.

To predict a satisfying safety profile of the uncovered compounds, off target activity and cellular toxicity were assayed. Potent compounds on both targets were tested for Kv7.3/5, Kv7.4 and hERG potential liability, potential liver toxicity, TRPV1 polymodality separation and specificity for TRPA1. The uncovered compounds exhibit improved tolerability profile, which is attributed mainly to the complete loss of hERG inhibition up to the maximal solubility of the compounds. In addition, some of the uncovered compounds show inhibition of capsaicin-induced activation of TRPV1, while not affecting either pH-induced or heat-induced activation of TRPV1. The selective inhibition of Capsaicin-gated TRPV1 currents, separated from additional inhibition of other TRPV1 activating modalities, is supportive of lack of hyperthermia liability. This TRPV1 polymodality separation was shown to be a good predictor of lack of thermal dysregulation in preclinical and clinical settings.

Identified compounds were progressed to in vitro DMPK studies to assess their potential druggability properties. Both LogD and human liver microsomes (HLM) were evaluated. Generally, the lower LogD values the compounds exhibit (i.e., more polar compounds), a lower HLM value is expected. Indeed, for most compounds with LogD<4, an HLM value <100 (L/min/mg) could be obtained. Other compounds show favorable metabolic stability. Good correlation was observed between acceptable HLM and rat liver microsomes (RLM), suggesting that similar metabolic pathways are involved in both species.

Using species-specific constructs and species-specific cells showed that no significant difference can be identified while comparing the inhibition-potency of the tested compounds either from human, rat or pig origin. The lack of a species differences in the inhibitory potency on TRPV1 suggests that rodent model could be used as predictor for efficacy of the uncovered compounds.

To study the inhibitory potency of the identified lead compounds on neuronal excitability in an experimental system of high physiological relevance, primary neonatal rat DRG neurons that comprise of the normal composition of neurons and glia were used. The inhibitory potency of the uncovered compounds was tested both on current-induced or capsaicin-induced neuronal activity.

It was shown that in current stimulation, the inhibitory potency of the tested compounds is mediated and identified through their effects on the voltage-activated effectors, such as the Kv7.2/3 target. In contrast, capsaicin-evoked responses identify the contribution of both the capsaicin-gated TRPV1 target, which depolarizes the membrane upon activation, and consequently activates the voltage-gated Kv7.2/3 target downstream.

Since neuronal excitability is a physiological predictive readout of, inter alia, pain propagation, the add-on effect of dual targeting both TRPV1 and Kv7.2/3 in a pain related system could be examined. It was found, for example, that a representative compound 421-6, with TRPV1 and Kv7.2/3 dual targeting properties (Kv7.2/3 EC50=2.1 μM; TRPV1 IC50=1.8 μM) at 2 μM, dramatically reduced the AP response to both current and capsaicin activation. Compound 421-6 displayed a <100 nM inhibition of capsaicin-induced neuronal firing. This high inhibition potency, significantly above the sum of inhibitions contributed by each target alone, indicates the synergistic effect of the exemplary compound 421-6. This synergism evolves the high inhibition potency of a respective compound and a superior specificity that emerges from a higher activity, which occurs only where both targets are co-expressed and their signaling pathways are crossing, the latter being unique to the nociceptive sensory neurons.

To provide additional physiological support in a human relevant system, human neural progenitor cells (hNPCs) that were differentiated to human sensory neurons were employed. Using spontaneous and induced current application methodologies it was demonstrated, similarly to the significant effect seen in rat DRGs, that the tested compounds have strong inhibition potency on the excitability of human sensory neurons. The superiority of the potency of the newly designed compounds compared to the standard of care drug used in clinics for neuropathic pain treatment, Gabapentin, was also demonstrated.

Embodiments of the present invention therefore relate to novel families of compounds derived from diphenylamine derivatives, which exhibit dual activity of opening a potassium channel (e.g., Kv7.2/3) and inhibiting TRPV1 activity, and which exhibit improved pharmacological profile, compared to previously disclosed diphenylamine derivatives; and to uses thereof in the treatment of medical conditions associated with these channels, particularly pain such as neuropathic pain.

### Compounds:

The newly designed compounds according to the present embodiments can be collectively represented by Formula I:

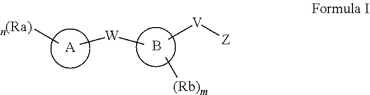

wherein:
A and B are each independently selected from an aryl and a heteroaryl;
W is selected from —S—, —O—, —CR₁(OH)—, —C(═O), and NRx, wherein Rx forms a nitrogen-containing heterocyclic ring with one of the Ra substituents of ring A;
n is an integer of from 1 to 5;
m is an integer of from 0 to 4;
Ra and Rb are each independently a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Ra substituents form together an alicyclic or heterocyclic ring, at least one of the Ra substituents forms together with Rx, if present, the nitrogen-containing heterocyclic ring and/or at least two of the Rb substituents form together an alicyclic or heterocyclic ring, wherein when n is greater than 1, each Ra is the same or different substituent, and when m is greater than 1, each Rb is the same or different substituent;
R₁, if present, is hydrogen or alkyl;
V is —(CR₂R₃)k-U;
k is 0, 1 or 2;
R₂ and R₃ are each independently hydrogen, alkyl, cycloalkyl and aryl;
U is amide (—C(═O)—NR₁₀—) or an isostere thereof; and
Z is represented by Formula II:

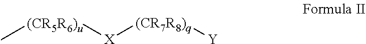

wherein:
u and q are each independently an integer of from 0 to 4, provided that u+q is at least 2;
X is selected from —O— and —NR₉—, or is absent;
Y is a polar hydrophilic group such as OR₁₁, SR₁₁, amine (NR₁₂R₁₃), or amide (—NR₁₂—C(═O)—R₁₄;
R₅, R₆, R₇ and R₈ are each independently selected from hydrogen, halo, alkyl, haloalkyl, cycloalkyl, heteroalicyclic, aryl, alkylamine, alkoxy, haloalkoxy, hydroxy, ether and aryloxy, or, alternatively, two of R₅, R₆, R₇, R₈ and R₉ form together an alicyclic or heteroalicyclic ring; and
R₉, R₁₀, R₁₁, R₁₂, R₁₃ and R₁₄ are each independently selected from hydrogen, alkyl, cycloalkyl, and aryl, or, alternatively, two of R₅, R₆, R₇, R₈, R₉ and R₁₁ or two of R₅, R₆, R₇, R₈, R₉, R₁₂ and R₁₃ form together an alicyclic or heteroalicyclic ring.

As discussed in the Examples section that follows, in a search for compounds with improved pharmacological profile, the present inventors have studied various manipulations to previously described compounds. These manipulations were performed on various parts of the skeleton of previously described compounds, namely, on rings A and B, as shown in Formula I, on the side chain denoted as —V—Z, particularly Z, in Formula I, and on the bridge between rings A and B, denoted W in Formula I. The present inventors have uncovered that the best performing compounds are those in which the amine bridge moiety in previously described compounds is replaced by other moieties, as defined herein for W in Formula I.

The following describes various embodiments of compounds of Formula I as described herein, regarding the A and B rings, the —V—Z side chain, and the W bridge.

### A and B Rings:

According to some of any of the embodiments described herein, A and B are each an aryl, as defined herein, and in some of these embodiments, A and B are each phenyl, and such compounds are collectively represented by Formula Ia:

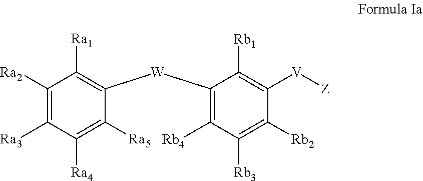

W, V and Z are as defined herein for Formula I;
Ra1-Ra5 are each independently as defined herein for Ra, and represent the substituents of Ring A; and
Rb1-Rb4 are each independently as defined herein for Rb, and represent the substituents of Ring B.

According to some of any of the embodiments described herein (e.g., for Formula I, Ia or any other relevant Formula), m is other than 0, and is 1, 2, 3 or 4, and at least one of the Rb substituent(s) is halo. Other substituents, if present (e.g., when m is 2, 3, or 4) are as defined herein for Rb.

According to some of any of the embodiments described herein (e.g., for Formula I, Ia or any other relevant Formula), m is 1. According to some of these embodiments, the Rb substituent is halo.

According to some of any of the embodiments described herein, m is 1 and the Rb substituent is at the para position with respect to the W, that is Rb2 in Formula Ta is a substituent and the other Rb substituents, for example, Rb1, Rb3 and Rb4, are each hydrogen. According to some of these embodiments, Rb in Formula I or Rb2 in Formula Ta is halo.

According to some of any of the embodiments described herein for Rb or Rb2 being halo, the halo is fluoro.

According to some of any of the embodiments described herein for Formula I, B is a heteroaryl. A can be an aryl or a heteroaryl and is preferably an aryl, for example, phenyl.

According to some of these embodiments, B is a nitrogen-containing heteroaryl as described herein, which can include, for example, 1, 2 or 3 nitrogen atoms within the ring, and in some embodiments, B is pyridine.

According to some of any of the embodiments described herein for Formula I, B is a heteroaryl, which can include, for example, 1, 2 or 3 nitrogen atoms within the ring, and A is an aryl, for example, phenyl.

According to some of any of the embodiments described herein for Formula I, B is pyridine and A is an aryl, for example, phenyl.

According to some of any of the embodiments described herein for Formula I, B is pyridine and A is phenyl, and such compounds are collectively represented by Formula Ib:

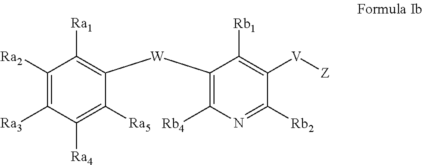

wherein:
W, V and Z are as defined herein for Formula I;
Ra1-Ra5 are each independently as defined herein for Ra; and
Rb1, Rb2 and Rb4 are each independently as defined herein for Rb.

According to some of any of the embodiments as described herein for Formula I, Ia, Tb and any other relevant formula as described herein, n is at least 3 and can be 3, 4 or 5, such that at least three positions of the A ring are substituted (by an Ra substituent that is other than hydrogen), or at least three of Ra1-Ra5 in Formula Ia or Tb are other than hydrogen.

According to some of any of the embodiments as described herein for Formula I, Ia, Tb and any other relevant formula as described herein, n is 3, such that three positions of the A ring are substituted (by an Ra substituent that is other than hydrogen), or three of Ra1-Ra5 in Formula Ta or Tb are other than hydrogen.

According to some of any of the embodiments as described herein for Formula I, Ta, Tb and any other relevant formula as described herein, two or more of the Ra substituents (e.g., at least two of Ra1-Ra5 in Formula Ta or Tb) are selected from halo and alkoxy.

According to some of these embodiments, two of the Ra substituents (e.g., two of Ra1-Ra5 in Formula Ta or Tb) are each independently alkoxy (which can be the same or different), two of the Ra substituents (e.g., two of Ra1-Ra5 in Formula Ta or Tb) are each independently halo (which can be the same or different), or one of the Ra substituents (e.g., one of Ra1-Ra5 in Formula Ta or Ib) is alkoxy and one of the Ra substituents (e.g., one of Ra1-Ra5 in Formula Ta or Tb) is halo.

According to some of any of the embodiments described herein, when one or more of the Ra substituents (e.g., one of Ra1-Ra5 in Formula Ta or Tb) is alkoxy, it is a lower alkoxy, of from 1 to 4 carbon atoms in length, for example, methoxy, ethoxy, propoxy, isopropoxy, amyloxy, butoxy, and/or isobutoxy, preferably methoxy.

According to some of these embodiments, two of the Ra substituents (e.g., two of Ra1-Ra5 in Formula Ta or Tb) are each independently halo, for example, chloro.

According to some of any of the embodiments as described herein for Formula I, Ia, Tb and any other relevant formula as described herein, at least one of the Ra substituent(s) (e.g., one of Ra1-Ra5 in Formula Ia or Tb) is alkyl, haloalkyl, cycloalkyl, aryl, alkoxy, alkylamine, heteroaryl or heteroalicyclic, or, alternatively, two Ra substituents ((e.g., two of Ra1-Ra5 in Formula Ta or Ib) form together a cyclic ring.

According to some of any of the embodiments as described herein for Formula I, Ta, Tb and any other relevant formula as described herein, at least one of the Ra substituent(s) (e.g., one of Ra1-Ra5 in Formula Ta or Tb) is alkyl, haloalkyl, cycloalkyl and/or aryl.

According to some of any of the embodiments as described herein for Formula I, Ta, Tb and any other relevant formula as described herein, at least one of the Ra substituent(s) (e.g., one of Ra1-Ra5 in Formula Ta or Tb) is alkyl or cycloalkyl.

According to some of any of the embodiments as described herein for Formula I, Ta, Tb and any other relevant formula as described herein, the Ra substituent at the ortho position with respect to W, namely Ra1 in Formula Ta or Tb, is other than hydrogen.

According to some of these embodiments, the Ra substituent at the ortho position with respect to W, namely Ra1 in Formula Ta or Tb, is alkyl, haloalkyl, cycloalkyl, aryl, alkoxy, alkylamine, heteroaryl or heteroalicyclic, as described herein in any of the respective embodiments, preferably alkyl or cycloalkyl.

According to some of any of the embodiments as described herein for Formula I, Ta, Tb and any other relevant formula as described herein, when one or more of the Ra substituent(s) (e.g., one of Ra1-Ra5 in Formula Ta or Tb) is alkyl, haloalkyl, cycloalkyl, aryl, alkoxy, alkylamine, heteroaryl or heteroalicyclic, as described herein in any of the respective embodiments, at least one such an Ra substituent is at the ortho position with respect to the W in Formula I.

According to some of any of the embodiments as described herein for Formula I, Ta, Tb and any other relevant formula as described herein, n is 3, one of the Ra substituent(s) is alkyl, haloalkyl, cycloalkyl, aryl, alkoxy, alkylamine, heteroaryl or heteroalicyclic, and is at the ortho position with respect to the W (namely it is Ra1 in Formula Ta or Ib), and the two other Ra substituents are each halo.

According to some of any of the embodiments as described herein for Formula Ta, Ib and any other relevant formula as described herein, n is 3, Ra1 is alkyl, haloalkyl, cycloalkyl, aryl, alkoxy, alkylamine, heteroaryl or heteroalicyclic, preferably alkyl or cycloalkyl, and two of the other Ra substituents (Ra2, Ra3, Ra4 and Ra5) are each halo, preferably chloro. According to some of these embodiments, Ra3 and Ra5 are each halo, preferably each is chloro.

According to some of any of the embodiments described herein for Formula I, n is 3; one of the Ra substituent(s) is alkyl, haloalkyl, cycloalkyl, aryl, alkoxy, alkylamine, heteroaryl or heteroalicyclic, preferably alkyl or cycloalkyl, and is at the ortho position with respect to the W; the two other Ra substituents are each halo, preferably chloro; m is 1; and Rb is halo, preferably chloro, and is at the para position with respect to the W.

According to some of any of the embodiments described herein for Formula Ia or Ib, Ra1 is alkyl, haloalkyl, cycloalkyl, aryl, alkoxy, alkylamine, heteroaryl or heteroalicyclic.

According to some of any of the embodiments described herein for Formula Ia or Ib, Ra3 and Ra5 are each independently a halo (e.g., chloro).

### —V—Z Side Chain:

According to some of any of the embodiments described herein, V is —(CR₂R₃)k-U; k is 0, 1 or 2; R₂ and R₃ are each independently hydrogen, alkyl, cycloalkyl and aryl; and U is amide (—C(═O)—NR₁₀—) or an isostere thereof.

According to some of any of the embodiments described herein, k is 1.

According to some of any of the embodiments described herein, k is 1, and R₂ and R₃ are each hydrogen.

According to exemplary embodiments, U is amide (—C(═O)—NR₁₀—, and in some of these embodiments, R₁₀ is hydrogen.

According to alternative embodiments, U is an amide isostere.

By “isostere” it is meant a moiety with similar shape and/or electronic properties and/or biological activity as the respective parent moiety (herein, amide), which is therefore expected to be recognized by the body similarly to the parent moiety. In the context of medicinal chemistry, isosteres are also referred to as bioisosteres.

Exemplary amide isosteres include, but are not limited to, sulfonamide, phosphonamidate, thioamide, ester, urea, fluoroalkene, alkene, tetrazole, oxadiazole, thiazole, triazole, imidazole, pyrazole, indole, pyridine, pyrazine, carbamate, amidine, trifluoroethylamine, hydroxyethyl, alpha-difluoroketone, and oxetanylamine.

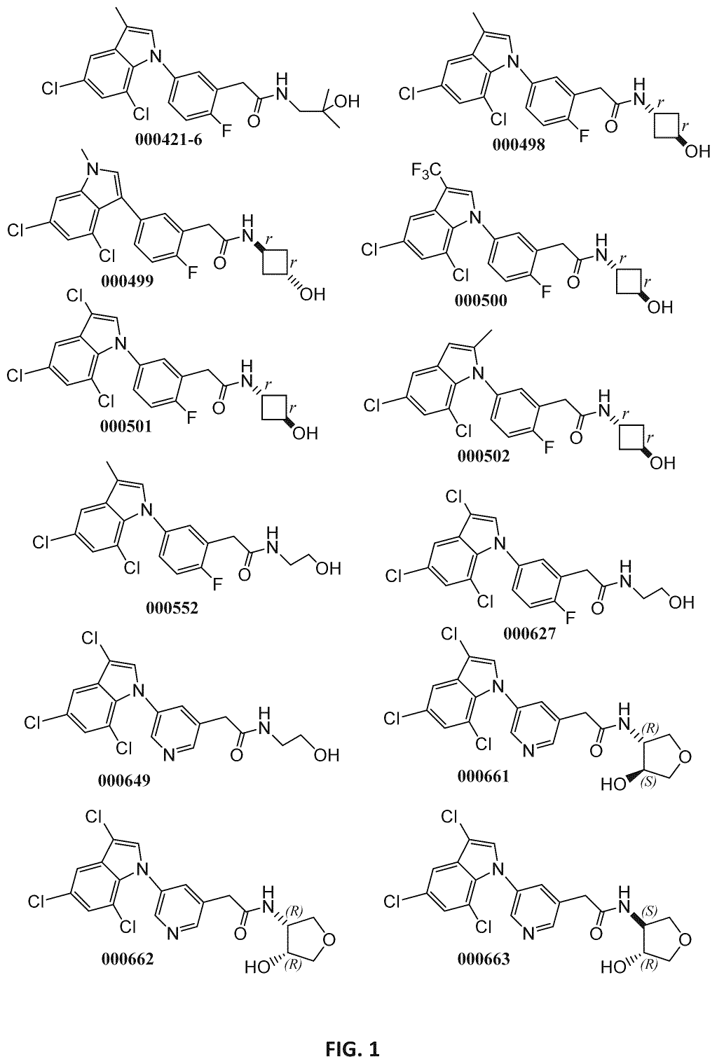

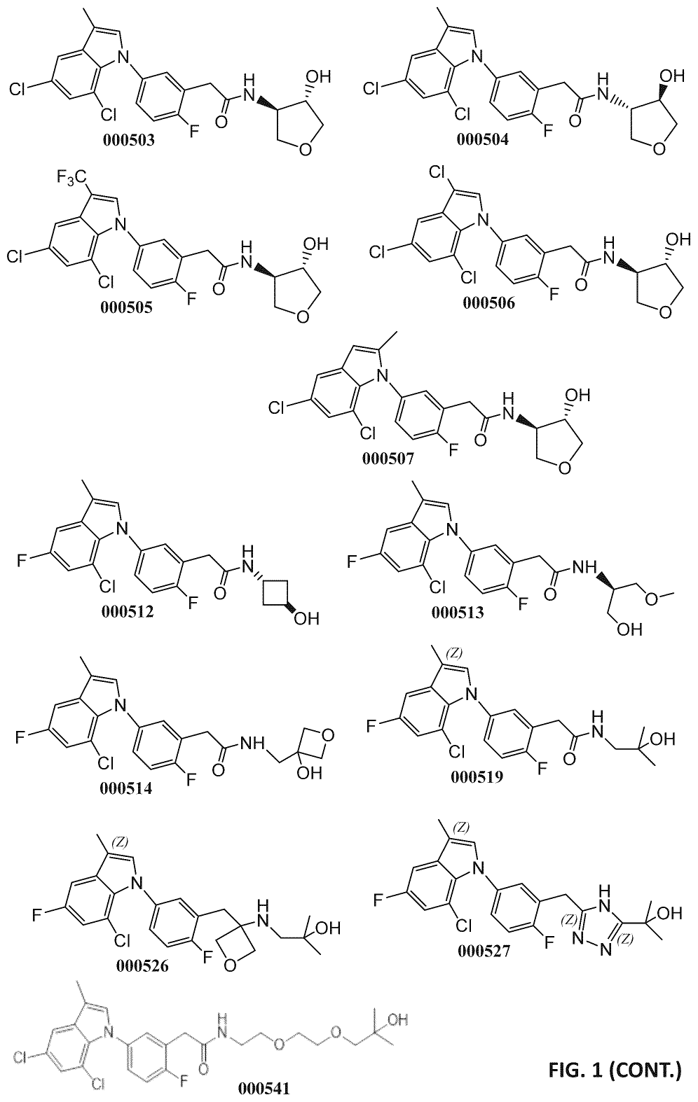

Exemplary isosteres are shown in FIG. 1, compound 526, for an oxetanylkamine, and compound 527, for triazole.

According to some of any of the embodiments described herein for Formula I, Ta, Ib and any other relevant formula, k is 1, R₂ and R₃ are each hydrogen, U is amide (—C(═O)—NR₁₀—) or an isostere thereof as described herein, and Z is represented by Formula II.

Moiety Z should preferably include one or more oxygen atoms, for example, as part of the side chain backbone (e.g., when X is O, or when Y is OR₁₁ and R₁₁ is other than hydrogen), as part of the terminal group of the side chain (e.g., when Y is OR₁₁ and R₁₁ is hydrogen, and/or as a substituent of the side chain backbone (e.g., when one or more of R₅, R₆, R₇ and R₈ is, or when two or more of R₅, R₆, R₇ and R₅, form together, an oxygen-containing heteroalicyclic (e.g., furan or oxetane) and/or one or more of R₅, R₆, R₇ and R₈ is hydroxy or alkoxy or an oxygen-containing heteroalicyclic as described herein, or a cycloalkyl substituted by hydroxy. Any combination of the foregoing is contemplated.

According to some of any of the embodiments described herein for Formula I, Ia and Ib and any other relevant formula, X is absent.

According to some of any of the embodiments described herein for Formula I, Ia and Ib and any other relevant formula, at least one of R₅, R₆, R₇ and R₈ is independently selected from alkyl, hydroxy, alkoxy, haloalkyl, ether, and halo, and/or at least two of R₅, R₆, R₇ and R₈ form together an alicyclic or heteroalicyclic ring.

According to some of any of the embodiments described herein for Formula I, Ia and Ib and any other relevant formula, two or more of R₅, R₆, R₇ and R₈ are independently selected from alkyl, haloalkyl and halo, and in some embodiments, two or more of R₅, R₆, R₇ and R₈ are each independently an alkyl.

According to some of any of the embodiments described herein for Formula I, Ia and Ib and any other relevant formula, u is 1 or 2. It is be noted that when u is greater than 1, and is composed of two or more (CR₅R₆) moieties, R₅ and R₆ is each moiety can be the same or different.

According to some of any of the embodiments described herein for Formula I, Ia and Ib and any other relevant formula, R₅ and R₆ are each hydrogen.

According to some of any of the embodiments described herein for Formula I, Ia and Ib and any other relevant formula, u is 1 or 2, and each of R₅ and R₆ is hydrogen.

According to some of any of the embodiments described herein for Formula I, Ia and Ib and any other relevant formula, q is 1 or 2. It is be noted that when q is greater than 1, and is composed of two or more (CR₇R₆) moieties, R₇ and R₈ is each moiety can be the same or different.

According to some of any of the embodiments described herein for Formula I, Ia and Ib and any other relevant formula, q is 1 and at least one or each of R₇ and R₈ is alkyl.

According to some of any of the embodiments described herein for Formula I, Ia and Ib and any other relevant formula, u is 1 or 2, each of R₅ and R₆ is hydrogen, q is 1 and at least one or each of R₇ and R₈ is alkyl. Exemplary such compounds include compounds 417, 417-4, 532, 270, 519, 421-6, 526, 527, as shown in FIGS. 1, 2 and 3A.

According to some of any of the embodiments described herein for Formula I, Ia and Ib and any other relevant formula, q is 1 and at least one or each of R₇ and R₈ is hydrogen.

According to some of any of the embodiments described herein for Formula I, Ia and Ib and any other relevant formula, u is 1 or 2, each of R₅ and R₆ is hydrogen, q is 1 and at least one or each of R₇ and R₈ is hydrogen. Exemplary such compounds include compounds 552 and 627, as shown in FIG. 1.

According to alternative embodiments of any of the embodiments described herein for Formula I, Ia and Ib and any other relevant formula, one or more of R₅, R₆, R₇ and R₈ is independently hydroxy, hydroxyalkyl, ether, a heteroalicyclic (e.g., oxygen-containing as described herein) or a cycloalkyl (e.g., of 3, 4 or 5 atoms). In some of these embodiments, the cycloalkyl is substituted by one or more hydroxy groups. According to some of these embodiments, u is 1 or 2. According to some of these embodiments, q is 1. According to some of these embodiments, u is 1 or 2 and q is 1. Exemplary such compounds include compounds 486, 490, 533, 536, 569, 573, 482, 513, 514, 414, 415, 482, as shown in FIGS. 1, 2, and 3A.

According to alternative embodiments of any of the embodiments described herein for Formula I, Ia and Ib and any other relevant formula, two or more of R₅, R₆, R₇ and R₈ form together an alicyclic ring, for example, cyclobutane. According to some of these embodiments, u is 1 or 2.

According to some of these embodiments, q is 1. According to some of these embodiments, u is 1 or 2 and q is 1. Exemplary such compounds include compounds 498, 499, 500, 501, 502, 512, 480, 481, 483, 484, 485, 489, 488, 534, 535, as shown in FIGS. 1, 2 and 3A.

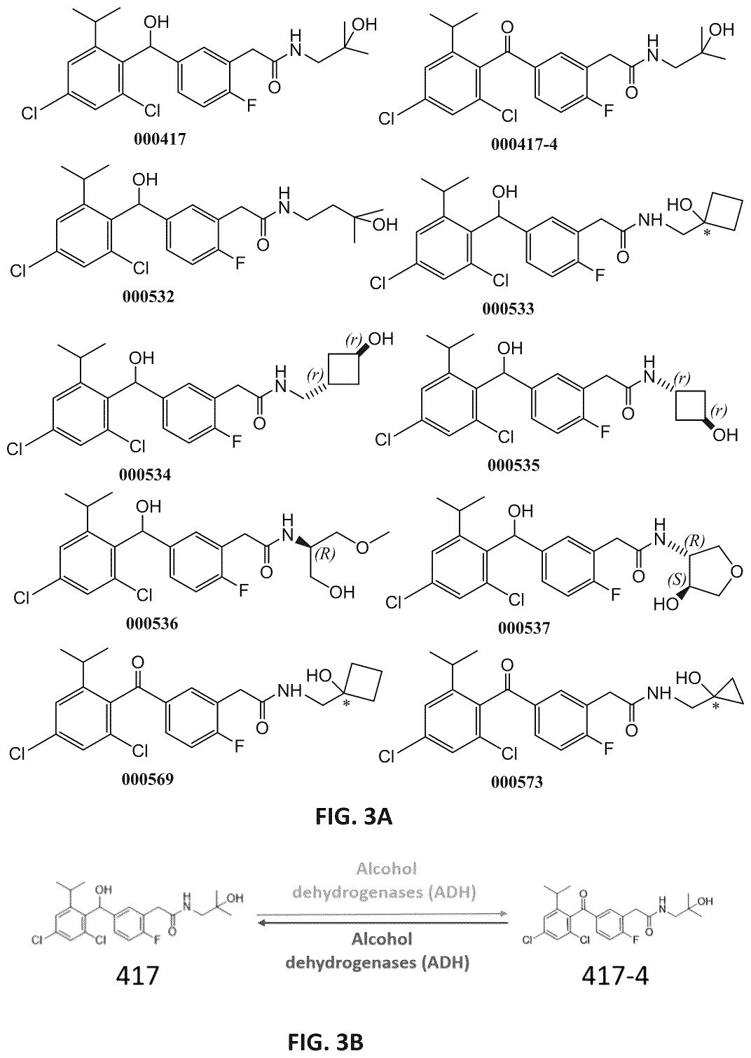

According to alternative embodiments of any of the embodiments described herein for Formula I, Ia and Ib and any other relevant formula, two or more of R₅, R₆, R₇ and R₈ form together a heteroalicyclic ring, preferably an oxygen-containing 4, 5 or 6-membered heteroalicyclic. In exemplary embodiments, the oxygen-containing heteroalicyclic ring is furan. According to some of these embodiments, u is 1 or 2. According to some of these embodiments, q is 1. According to some of these embodiments, u is 1 or 2 and q is 1. According to some of any of these embodiments, X is absent. Exemplary such compounds include compounds 503, 504, 505, 506, 507, 537, as shown in FIGS. 1 and 3A.

According to some of any of the embodiments described herein for Formula I, Ia and Ib and any other relevant formula, in combination with any of the embodiments described herein for Formula II, Y is OR₁₁, and R₁₁ is hydrogen.

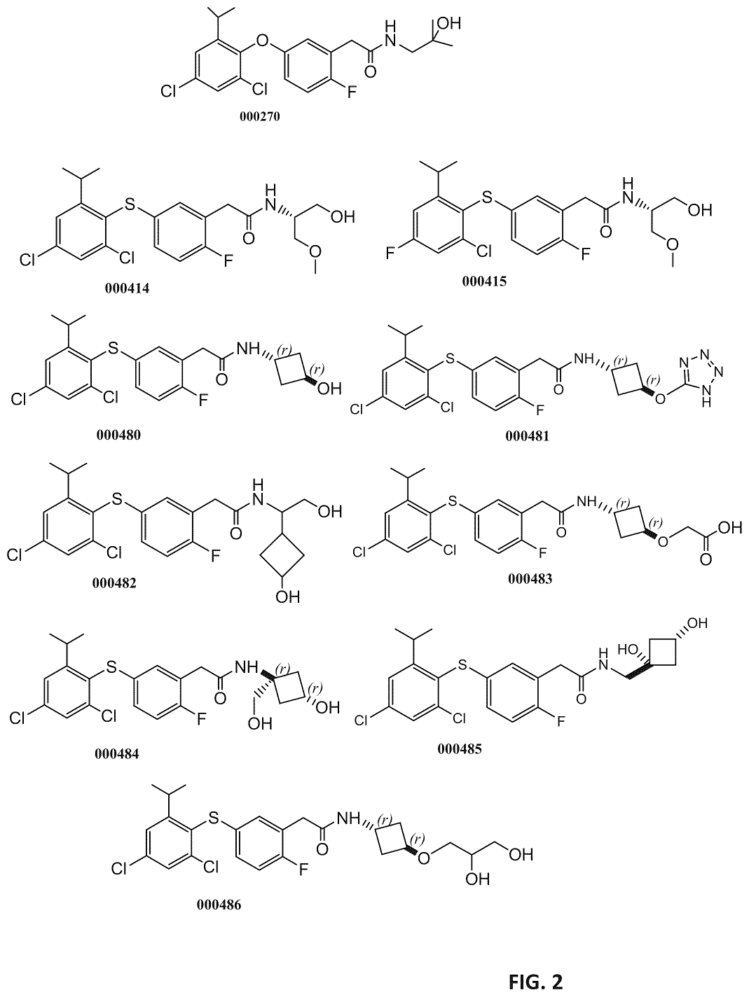

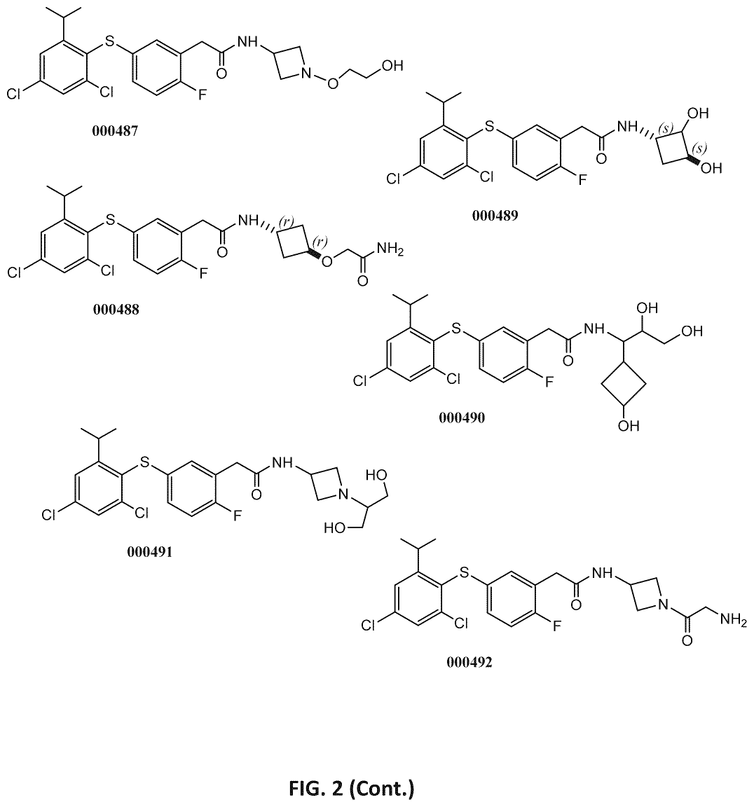

According to some of any of the embodiments described herein for Formula I, Ia and Ib and any other relevant formula, in combination with any of the embodiments described herein for Formula II, Y is OR₁₁ and R₁₁ is an alkyl, preferably substituted by at least one hydroxy, amide and/or carboxy. Exemplary such compounds include compounds 486, 483 and 488, as shown in FIGS. 1 and 2.

According to some of any of the embodiments described herein for Formula I, Ia and Ib and any other relevant formula, in combination with any of the embodiments described herein for Formula II, Y is OR₁₁ and R₁₁ is a heteroaryl or heteroalicyclic, as defined herein. An exemplary such a compound is compound 481 (see, FIG. 2).

According to some of any of the embodiments described herein for Formula I, Ia and Ib and any other relevant formula, in combination with any one of the embodiments described herein for Formula II, Y is NR₁₂R₁₃.

According to some of any of the embodiments described herein for Formula I, Ia and Ib and any other relevant formula, in combination with any one of the embodiments described herein for Formula II, Y is NR₁₂R₁₃, and R₁₂ forms together with one or more of R₅, R₆, R₇ and R₈ a nitrogen-containing heteroalicyclic (e.g., azetidine). In some of these embodiments, R₁₃ is hydrogen, hydroxy, alkyl, hydroxyalkyl, alkylene glycol or alkoxy. Exemplary such compounds include compounds 487 and 491, as shown in FIG. 2.

According to some of any of the embodiments described herein for Formula I, Ia and Ib and any other relevant formula, in combination with any one of the embodiments described herein for Formula II, Y is —NR₁₂—C(═O)—R₁₄.

According to some of any of the embodiments described herein for Formula I, Ia and Ib and any other relevant formula, in combination with any one of the embodiments described herein for Formula II, Y is —NR₁₂—C(═O)—R₁₄, and R₁₂ forms together with one or more of R₅, R₆, R₇ and R₈ a nitrogen-containing heteroalicyclic (e.g., azetidine). An exemplary such a compound is compound 492, as shown in FIG. 2.

### W Bridge:

According to some of any of the embodiments described herein for Formulae I, Ia, Ib and II, and any combination thereof, W is —NRx- and Rx forms together with one of the Ra substituents a nitrogen-containing heterocyclic ring. The nitrogen-containing heterocyclic ring can be a 4-, 5-, 6-, 7-, or 8-membered ring, preferably 4-, 5- or 6-membered ring. The heterocyclic ring can be a heteroaryl or a heteroalicyclic, fused to ring A.

According to some of these embodiments, Rx forms the heterocyclic ring with an Ra substituent at the ortho position with respect to W, as described herein (e.g., Ra1 in Formula Ia or Ib). Such compounds are also referred to herein as Chemotype I compounds.

According to some of the respective embodiments of Formula I, A is aryl (e.g., phenyl) and B is aryl (e.g., phenyl) or heteroaryl. In some embodiments, when B is a heteroaryl, the heteroaryl is a nitrogen-containing heteroaryl, which can include 1, 2 or 3 nitrogen atoms within the ring. In some embodiments, when B is a heteroaryl, the heteroaryl is pyridine.

According to some of any of the embodiments described herein for compounds of Chemotype I, the compounds are collectively represented by Formula III:

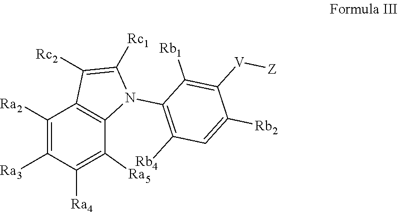

wherein:
—V—Z (the side chain moiety) is as described herein in any of the respective embodiments and any combination thereof;
D is N or C—Rb3;
Ra2-Ra5, Rb1-Rb4, V and Z are as defined and described herein for Formula I, Ia or Ib, in any of the respective embodiments and any combination thereof; and
Rc1 and Rc2 are each independently a substituent as defined herein for Ra1-Ra5 in any of the respective embodiments and any combination thereof.

According to these embodiments, Rx and Ra1 form together a nitrogen-containing heteroaryl that is fused to ring A.

According to some of any of the embodiments described herein for Formula III, Rc1 and Rc2 can each independently be hydrogen, or a substituent such as alkyl, haloalkyl, and/or halo. In exemplary embodiments, Rc1 is hydrogen and Rc2 is alkyl, preferably a lower alkyl of 1-4 carbon atoms in length, for example, methyl; or haloalkyl, for example, trihaloalkyl such as CF₃, or halo, for example, fluoro or chloro.

Exemplary compounds of Formula III, Chemotype I, are presented in FIG. 1.

According to some of any of the embodiments described herein for Formulae I, Ia, Ib and II, and any combination thereof, W is —S—. Such compounds are also referred to herein as Chemotype II compounds.

According to some of the respective embodiments of Formula I, A is aryl (e.g., phenyl) and B is aryl (e.g., phenyl) or heteroaryl such as a nitrogen-containing heteroaryl as described herein (e.g., pyridine).

According to some of any of the embodiments described herein for compounds of Chemotype II, the compounds are collectively represented by Formula IV:

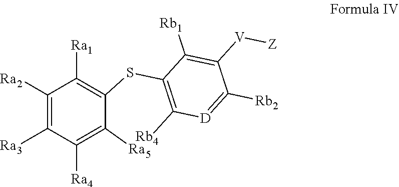

wherein:
—V—Z (the side chain moiety) is as described herein in any of the respective embodiments and any combination thereof;
D is N or C—Rb3; and
Ra1-Ra5, Rb1-Rb4, V and Z are as defined herein for Formula I, Ia or Ib in any of the respective embodiments and any combination thereof.

According to exemplary embodiments, Ra1 is alkyl or cycloalkyl, as described herein for Formula I, Ia or Ib.

Exemplary compounds of Formula IV, Chemotype II, are presented in FIG. 2.

According to some of any of the embodiments described herein for Formulae I, Ia, Ib and II, and any combination thereof, W is —CR₁(OH)—, and R₁ is as defined herein. Such compounds are also referred to herein as Chemotype III compounds.

According to some of the respective embodiments of Formula I, A is aryl (e.g., phenyl) and B is aryl (e.g., phenyl) or heteroaryl such as a nitrogen-containing heteroaryl as described herein (e.g., pyridine).

According to some of any of the embodiments described herein for compounds of Chemotype III, the compounds are collectively represented by Formula V:

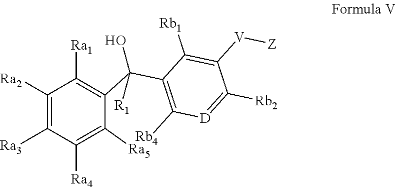

wherein:
—V—Z (the side chain moiety) is as described herein in any of the respective embodiments and any combination thereof;
D is N or C—Rb3; and
Ra1-Ra5, Rb1-Rb4, R₁, V and Z are as defined herein for Formula I, Ia or Ib, according to any of the respective embodiments and any combination thereof.

According to exemplary embodiments, Ra1 is alkyl or cycloalkyl, as described herein for Formula I, Ta or Ib.

According to some of any of the embodiments described herein for Formulae I, Ta, Ib and II, and any combination thereof, W is —C(═O)—. Such compounds are also referred to herein as Chemotype IV compounds.

According to some of the respective embodiments of Formula I, A is aryl (e.g., phenyl) and B is aryl (e.g., phenyl) or heteroaryl (e.g., pyridine).

According to some of any of the embodiments described herein for compounds of Chemotype IV, the compounds are collectively represented by Formula VI:

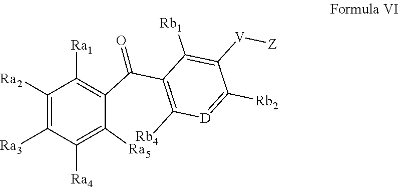

wherein:
—V—Z (the side chain moiety) is as described herein in any of the respective embodiments and any combination thereof;
D is N or C—Rb3; and
Ra1-Ra5, Rb1-Rb4, V and Z are as defined herein for Formula I, Ia or Ib.

According to exemplary embodiments, Ra1 is alkyl or cycloalkyl, as described herein for Formula I, Ia or Ib.

Exemplary compounds of Formulae V and VI, Chemotype III and IV, are presented in FIG. 3A. As discussed in the Examples section that follows and shown in FIG. 3B, Chemotype III compounds can be converted enzymatically to Chemotype IV compounds and vice versa.

According to some of any of the embodiments described herein, the newly designed compounds are such that exhibit one or more, or two or more, or three or more, and preferably all of the following characteristics:
LogD, determined as described herein, higher than 4, or higher than 5, or ranging between 4 and 7;
Ligand-lipophilicity efficiency (LLE), determined as described herein, higher than 3, or higher than 5;
HLM Clint, determined as described herein, lower than 100 μl/min/mg; and
Kinetic solubility higher than 20 or higher than 30 micromolar.

For any of the embodiments described herein, and any combination thereof, the compound may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., amine and/or amide and/or a nitrogen atom in a heterocyclic group) group of the compound which is in a positively charged form (e.g., wherein the basic group is protonated), in combination with at least one counter-ion, derived from the selected acid, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment, which by itself has handedness, such as all living systems. In the context of the present embodiments, a compound may exhibit one or more chiral centers, each of which exhibiting an R—or an S-configuration and any combination, and compounds according to some embodiments of the present invention, can have any their chiral centers exhibit an R—or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter, they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. Exemplary prodrugs of compounds according to some of the present embodiments include esters of a hydroxy group (e.g., in the side chain), including carboxylic esters, phosphate esters, and the like.

The term “solvate” refers to a complex of variable stoichiometry (e.g., di-, tri-, tetra-, penta-hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

### Pharmaceutical Composition:

In any of the methods and uses described herein, the compounds of the present embodiments can be utilized per se, or in a pharmaceutical composition where it is mixed with suitable carriers or excipients.

According to an aspect of some embodiments of the present invention there is provided a pharmaceutical composition comprising the compound as described herein and a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compound or combination of compounds which are accountable for the biological effect.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

According to some embodiments of the present invention, a compound or pharmaceutical composition as described herein are administered topically.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, nonirritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include crosslinked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

According to some embodiments of the present invention, the compound or pharmaceutical composition as described herein are administered so as to deliver the compound to the central and/or peripheral nervous system.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Local administration by perineural injection, or by means of eye or ear drops, is also contemplated for delivering the active compounds to the central nervous system.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of the active ingredient (a compound as described herein) effective to prevent, alleviate or ameliorate symptoms of a medical condition as described herein or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue (e.g., plasma) levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine concentrations in the relevant tissue (e.g., plasma and/or brain).

In some embodiments of any of the embodiments described herein, an effective amount of the compound is less than 100 μM. In some embodiments, an effective amount is less than 10 μM. In some embodiments, an effective amount is less than 5 μM. In some embodiments, an effective amount is less than 1 μM. In some embodiments, an effective amount is less than 0.5 μM. In some embodiments, an effective amount is less than 0.1 μM.

In some embodiments of any of the embodiments described herein, an effective amount of the compound ranges from 1 μM to 1 mM, or from 1 μM to 100 μM, or from 100 μM to 100 μM, or from 100 μM to 10 μM, or from 100 μM to 1 μM, or from 100 μM to 500 nM, or from 100 μM to 100 nM, including any intermediate values and subranges therebetween.

In some embodiments of any of the embodiments described herein, an effective amount is at least 100% of the IC50 of the compound towards TRPV1 and/or Kv7.2/3. In some embodiments, an effective amount is at least 200% of the IC50 of the compound towards TRPV1 and/or Kv7.2/3. In some embodiments, an effective amount is at least 300% of the IC50 of the compound towards TRPV1 and/or Kv7.2/3. In some embodiments, an effective amount is at least 500% of the IC50 of the compound towards TRPV1 and/or Kv7.2/3. In some embodiments, an effective amount is at least 1000% of the IC50 of the compound towards TRPV1 and/or Kv7.2/3.

In some embodiments of any of the embodiments described herein, an effective amount of a compound as described herein is at the nM range (e.g., from 0.001 to 1,000 nM, or from 0.001 nM to 100 nM).

In some embodiments of any of the embodiments described herein, an effective amount of a compound as described herein is lower by at least 10%, or by at least 20, 30, 40, 50, 60, 70, 80, 90, 100%, or even more, than an amount that causes hERG inhibition.

In some embodiments of the present invention, the amount of the compound or pharmaceutical composition to be administered required to achieve a therapeutic effect (e.g., a dosage or a therapeutically effective amount of the compound as described herein) is lower than an amount of previously described compounds known to exhibit the same therapeutic effect by at least 20%, or at least 30%.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed herein.

As used herein, the term “subject” includes mammals, preferably human beings at any age which suffer from the pathology. Preferably, this term encompasses individuals who are at risk to develop the pathology.

It will be appreciated that the compounds or pharmaceutical compositions as described herein can be provided alone or in combination with other active ingredients, which are well known in the art for alleviating the medical condition (e.g., neuropathic pain) and/or for activating a potassium channel as described herein and/or for inhibiting an activity of TRPV1.

In some embodiments, the compounds or pharmaceutical compositions as described herein may be administered with an activator of a potassium channel as described herein (e.g., Kv7.2/3), either together in a co-formulation or in separate formulations.

The pharmaceutical composition may further comprise additional pharmaceutically active or inactive agents such as, but not limited to, an anti-bacterial agent, an antioxidant, a buffering agent, a bulking agent, a surfactant, an anti-inflammatory agent, an anti-viral agent, a chemotherapeutic agent and an anti-histamine, and/or an additional agent usable in treating a medical condition, disease or disorder as described herein.

### Uses:

According to an aspect of some embodiments of the present invention there is provided a compound as described herein or a pharmaceutical composition as described herein, for use in modulating an activity of a voltage-dependent potassium channel.

According to an aspect of some embodiments of the present invention there is provided a method of modulating an activity of a voltage-dependent potassium channel, which comprises contacting the potassium channel with a compound or a pharmaceutical composition as described herein. The contacting can be effect in vitro, e.g., by contacting a cell, a tissue or an organ which express the channel with the compound or composition, or in vivo, by administering to a subject in need thereof a therapeutically effective amount of the compound or composition.

In some embodiments, the potassium channel is Kv7.2/7.3 (which is also referred to herein interchangeably as Kv7.2/3).

In some embodiments, the modulating comprises opening the potassium channel.

According to an aspect of some embodiments of the present invention there is provided a compound as described herein or a pharmaceutical composition as described herein, for use in modulating an activity of a TRPV1 channel.

According to an aspect of some embodiments of the present invention there is provided a method of modulating an activity of a TRPV1 channel, which comprises contacting the TRPV1 channel with a compound or a pharmaceutical composition as described herein. The contacting can be effect in vitro, e.g., by contacting a cell, a tissue or an organ which express the channel with the compound or composition, or in vivo, by administering to a subject in need thereof a therapeutically effective amount of the compound or composition.

In some embodiments, the modulating comprises inhibiting the activity of the TRPV1 channel (e.g., blocking the channel).

According to an aspect of some embodiments of the present invention there is provided a compound as described herein or a pharmaceutical composition as described herein, for use in modulating an activity of both a voltage-dependent potassium channel and a TRPV1 channel, as described herein in any of the respective embodiments.

According to an aspect of some embodiments of the present invention there is provided a method of modulating an activity of a voltage-dependent potassium channel and of a TRPV1 channel, which comprises contacting these channels with a compound or a pharmaceutical composition as described herein. The contacting can be effect in vitro, e.g., by contacting a cell, a tissue or an organ which express these channels with the compound or composition, or in vivo, by administering to a subject in need thereof a therapeutically effective amount of the compound or composition.

According to an aspect of some embodiments of the present invention there is provided a compound as described herein or a pharmaceutical composition as described herein, for use in treating a medical condition associated with an activity of a voltage-dependent potassium channel and/or of a TRPV1 channel.

According to an aspect of some embodiments of the present invention there is provided a compound as described herein or a pharmaceutical composition as described herein, for use in treating a medical condition associated with an activity of a voltage-dependent potassium channel and of a TRPV1 channel.

According to an aspect of some embodiments of the present invention there is provided a method of treating a medical condition associated with an activity of a voltage-dependent potassium channel and/or of a TRPV1 channel in a subject in need thereof, the method comprising administering to the subject a therapeutically effective amount of a compound or a pharmaceutical composition as described herein in any of the respective embodiments and any combination thereof.

According to some of any of the embodiments described herein, the medical condition is such that modulating an activity of one, and preferably both of a voltage-dependent potassium channel and a TRPV1 channel, as described herein, is beneficial.

According to some of any of the embodiments described herein, the medical condition is such that opening a voltage-dependent potassium channel and in habiting an activity (e.g., blocking) a TRPV1 channel, as described herein, is beneficial.

An exemplary medical condition is neuropathic pain.

Any other medical conditions (pathologies, conditions, diseases and/or disorders) that are associated with TRPV1 channel functioning and/or a voltage-dependent potassium channel as described herein are contemplated.

Exemplary medical conditions that are beneficially treatable by the TRPV1 inhibitors (e.g., blockers) described herein (compounds having general Formula I) include, but are not limited to, epilepsy, pain related conditions such as neurogenic pain, neuropathic pain, allodynia, pain associated with inflammation, and pain associated with pancreatitis, bipolar disorder, mood disorder, psychotic disorder, schizophrenia, anxiety, tinnitus and a motor neuron disease, bladder overactivity, urinary incontinence, persistent visceral hypersensitivity, including irritable bowel syndrome (IBD), chronic cough, and cancer (for example, squamous cell carcinoma, prostate carcinoma and pancreatic cancer).

There is much pathology, conditions and disorders that is associated with defective potassium channel functioning. Just as other potassium channel opening compounds, the compounds described herein are for use within the framework of a treatment for pathologies, conditions, disease and disorders associated with defective potassium channel functioning, so as to treat, ameliorate, prevent, inhibit, or limit the effects of the conditions and pathologies in animals including humans.

Exemplary medical conditions that are beneficially treatable by the potassium channel openers described herein include, but are not limited to, central or peripheral nervous system disorders such as ischemic stroke, migraine, ataxia, Parkinson's disease, bipolar disorders, trigeminal neuralgia, spasticity, mood disorders, brain tumors, psychotic disorders, schizophrenia, pruritus, myokymia, neurogenic pain, neuropathic pain, seizures, epilepsy, tinnitus, hearing and vision loss, anxiety and motor neuron diseases. The compounds described herein can further be beneficially used as neuroprotective agents (e.g., to prevent stroke and the like). The compounds described herein are also useful in treating disease states such as gastroesophogeal reflux disorder and gastrointestinal hypomotility disorders.

The compounds disclosed herein can also be used as potent candidates for treating a variety of medical conditions wherein depressing the cortical and/or peripheral neuron activity is beneficial, such as, for example, epilepsy, ischemic stroke, migraine, ataxia, myokymia, neurogenic pain, neuropathic pain, Parkinson's disease, bipolar disorder, trigeminal neuralgia, spasticity, mood disorder, psychotic disorder, schizophrenia, brain tumor, hearing and vision loss, anxiety, tinnitus and a motor neuron disease.

According to an aspect of some embodiments of the present invention, the compound or the composition as described herein is for use in depressing a cortical and/or peripheral neuron activity and/or in treating a condition in which depressing a cortical and/or peripheral neuron activity in a subject is beneficial, as described herein.

The compounds disclosed herein are particularly usable for treating medical conditions associated with neuronal hyperexcitability.

According to an aspect of some embodiments of the present invention, the compound or the composition as described herein is for use in treating a medical condition associated with hyperexcitability in a subject in need thereof.

According to an aspect of some embodiments of the present invention, the compound or the composition as described herein is for use in the preparation of a medicament for treating a medical condition associated with hyperexcitability in a subject in need thereof.

According to an aspect of some embodiments of the present invention, there is provided a method of treating a medical condition associated with hyperexcitability in a subject in need thereof, which is effected by administering to the subject a therapeutically effective amount of a compound or a composition as described herein in any of the respective embodiments and any combination thereof.

Medical conditions associated with neuronal hyperexcitability include, but are not limited to, epilepsy, neurodegeneration, neurodevelopmental disorders, Stroke, retinal degeneration, tinnitus, spinal cord injury, traumatic brain injury, hearing loss (through noise overexposure or ototoxicity), neuropathic pain, attention deficit hyperactivity disorder, autism, central pain syndromes, neurodegenerative diseases, multiple sclerosis, Alzheimer's disease, amyotrophic lateral sclerosis (ALS), Parkinson's disease, frontotemporal dementia, schizophrenia, Rasmussen's encephalitis, Huntington's disease, alcoholism or alcohol withdrawal, over-rapid benzodiazepine withdrawal, neonatal convulsions, episodic ataxia, myokymia, cerebral ischemia, cerebral palsy, asphyxia, anoxia, prolonged cardiac surgery, hypoglycemia, AIDS related dementia and anxiety disorders.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it means that the group, in this case the alkyl group, may contain 1 carbon atom, 2 carbon atoms, 3 carbon atoms, etc., up to and including 20 carbon atoms. More preferably, it is a medium size alkyl having 1 to 10 carbon atoms. Most preferably, it is a lower alkyl having 1 to 4 carbon atoms. The alkyl group may be substituted or unsubstituted. When substituted, the substituent group can be, for example, cycloalkyl, aryl, heteroaryl, heteroalicyclic, hydroxy, alkoxy, aryloxy, thiohydroxy, thioalkoxy, thioaryloxy, cyano, halo, carbonyl, thiocarbonyl, O-carbamyl, N-carbamyl, O-thiocarbamyl, N-thiocarbamyl, C-amido, N-amido, C-carboxy, O-carboxy, nitro, sulfonamido, trihalomethanesulfonamido, silyl, guanyl, guanidino, ureido, amino or NR′R″, wherein R′ and R″ are each independently hydrogen, alkyl, cycloalkyl, aryl, carbonyl, sulfonyl, trihalomethysulfonyl and, combined, a five- or six-member heteroalicyclic ring.

A “haloalkyl” groups describes an alkyl, as defined herein, substituted by one or more halo substituents, as defined herein. In some embodiments, the haloalkyl is an alkyl substituted by two or more, or three of more, halo substituents. In some embodiments, each of the halo substituents is fluoro. In some embodiments, a haloalkyl is —CF₃ or —CF₂H.

A “cycloalkyl” group refers to an all-carbon monocyclic or fused ring (i.e., rings which share an adjacent pair of carbon atoms) group wherein one of more of the rings does not have a completely conjugated pi-electron system (an alicyclic ring). Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, aryl, heteroaryl, heteroalicyclic, hydroxy, alkoxy, aryloxy, thiohydroxy, thioalkoxy, thioaryloxy, cyano, halo, carbonyl, thiocarbonyl, C-carboxy, O-carboxy, O-carbamyl, N-carbamyl, C-amido, N-amido, nitro, amino and NR′R″ as defined herein.

An “alkenyl” group refers to an alkyl group, which consists of at least two carbon atoms and at least one carbon-carbon double bond.

An “alkynyl” group refers to an alkyl group, which consists of at least two carbon atoms and at least one carbon-carbon triple bond.

In some embodiments, whenever an alkyl substituent is indicated, it can be replaced by an alkynyl or an alkynyl, as defined herein.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, halo, trihalomethyl, alkyl, hydroxy, alkoxy, aryloxy, thiohydroxy, thiocarbonyl, C-carboxy, O-carboxy, O-carbamyl, N-carbamyl, O-thiocarbamyl, N-thiocarbamyl, C-amido, N-amido, sulfinyl, sulfonyl, amino and NR′R″ as defined herein.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, cycloalkyl, halo, trihalomethyl, hydroxy, alkoxy, aryloxy, thiohydroxy, thiocarbonyl, sulfonamido, C-carboxy, O-carboxy, sulfinyl, sulfonyl, O-carbamyl, N-carbamyl, O-thiocarbamyl, N-thiocarbamyl, C-amido, N-amido, amino or NR′R″ as defined herein.

Herein throughout, and, for example, in Formulae II, III, IV and V, whenever pyridine is described as a heteroaryl B ring, it is to be noted that other nitrogen-containing heteroaryls are also contemplated.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, alkyl, cycloalkyl, aryl, heteroaryl, halo, trihalomethyl, hydroxy, alkoxy, aryloxy, thiohydroxy, thioalkoxy, thioaryloxy, cyano, nitro, carbonyl, thiocarbonyl, C-carboxy, O-carboxy, O-carbamyl, N-carbamyl, O-thiocarbamyl, N-thiocarbamyl, sulfinyl, sulfonyl, C-amido, N-amido, amino and NR′R″ as defined above.

As used herein, a “cyclic group” describes an alicyclic group (a cycloalkyl), an aryl, a heteroaryl or an heteroalicyclic.

A “hydroxy” group refers to an —OH group.

An “azido” group refers to a —N═N group.

An “alkoxy” group refers to both an —O-alkyl and an —O-cycloalkyl group, as defined herein.

A “haloalkoxy” group describes an O-alkyl group where the alkyl is a haloalkyl as described herein.

An “aryloxy” group refers to both an —O-aryl and an —O-heteroaryl group, as defined herein.

A “thiohydroxy” or “thiol” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, cycloalkyl, aryl, heteroaryl (bonded through a ring carbon) or heteroalicyclic (bonded through a ring carbon) as defined herein.

An “aldehyde” group refers to a carbonyl group, where R′ is hydrogen.

A “thiocarbonyl” group refers to a —C(═S)—R′ group, where R′ is as defined herein.

The term “carboxylate” encompasses C-carboxylate and O-carboxylate.

A “C-carboxy” group refers to a —C(═O)—O—R′ groups, where R′ is as defined herein.

An “O-carboxy” group refers to an R′C(═O)—O—group, where R′ is as defined herein.

A “carboxylic acid” group refers to a C-carboxyl group in which R′ is hydrogen.

A “halo” group refers to fluorine, chlorine, bromine or iodine.

A “trihalomethyl” group refers to a —CX₃ group wherein X is a halo group as defined herein.

A “trihalomethanesulfonyl” group refers to an X₃CS(═O)₂—group wherein X is a halo group as defined herein.

A “sulfinyl” group refers to an —S(═O)—R′ group, where R′ is as defined herein.

A “sulfonyl” group refers to an —S(═O)₂—R′ group, where R′ is as defined herein.

The term “sulfonylamide” encompasses S-sulfonylamide and N-sulfonylamido.

An “S-sulfonamido” group refers to a —S(═O)2-NR′R″ group, with R′ is as defined herein and R″ is as defined for R′.

An “N-sulfonamido” group refers to an R'S(═O)₂—NR″ group, where R′ and R″ are as defined herein.

A “trihalomethanesulfonamido” group refers to an X₃CS(═O)₂NR′—group, where R′ and X are as defined herein.

The term “carbamate” encompasses O-carbamyl and N-carbamyl.

An “O-carbamyl” group refers to an —OC(═O)—NR′R″ group, where R′ and R″ are as defined herein.

An “N-carbamyl” group refers to an R′OC(═O)—NR″—group, where R′ and R″ are as defined herein.

The term “thiocarbamate” encompasses O-thiocarbamyl and N-thiocarbamyl.

An “O-thiocarbamyl” group refers to an —OC(═S)—NR′R″ group, where R′ and R″ are as defined herein.

An “N-thiocarbamyl” group refers to an R″OC(═S)NR′—group, where R′ and R″ are as defined herein.

An “amino” group refers to an —NR′R″ group, where R′ and R″ are as defined herein.

An “alkylamino” group refers to an amine group is which one of R′ and R″ is alkyl (monoalkylamine) or in which both R′ and R″ are each independently an alkyl (dialkylamine).

The term “amide” encompasses C-amido and N-amido.

A “C-amido” group refers to a —C(═O)—NR′R″ group, where R′ and R″ are as defined herein.

An “N-amido” group refers to an R′C(═O)—NR″ group, where R′ and R″ are as defined herein.

A “quaternary ammonium” group refers to an —NHR′R″⁺ group, wherein R′ and R″ are independently alkyl, cycloalkyl, aryl or heteroaryl.

An “ureido” group refers to an —NR′C(═O)—NR″R′″ group, where R′ and R″ are as defined herein and R′″ is defined as either R′ or R″.

A “guanidino” group refers to an —R′NC(═N)—NR″R′″ group, where R′, R″ and R′″ are as defined herein.

A “guanyl” group refers to an R′R″NC(═N)—group, where R′ and R″ are as defined herein.

A “nitro” group refers to an —NO₂ group.

A “cyano” group refers to a —C≡N group.

A “silyl” group refers to a —SiR′R″R′″, where R′, R″ and R′″ are as defined herein.

As used herein, the term “alkylene glycol” describes a —O—[(CR′R″)_{z}—O]_{y}—R′″ end group or a —O—[(CR′R″)_{z}—O]_{y}—linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo(alkylene glycol).

A “leaving group” as used herein and in the art describes a labile atom, group or chemical moiety that readily undergoes detachment from an organic molecule during a chemical reaction, while the detachment is typically facilitated by the relative stability of the leaving atom, group or moiety thereupon. Typically, any group that is the conjugate base of a strong acid can act as a leaving group. Representative examples of suitable leaving groups according to some of the present embodiments include, without limitation, trichloroacetimidate, acetate, tosylate, triflate, sulfonate, azide, halide (halo, preferably bromo or iodo), hydroxy, thiohydroxy, alkoxy, cyanate, thiocyanate, nitro and cyano.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

### Solubility Assay:

Solubility measurements were performed prior to each in vitro assay to ensure accurate dose response and reproducibility. Briefly, each compound was dissolved to 60 mM in DMSO and calibration curve was performed from 1.5-200 μM using absorbance spectra analysis and determination of λmax. Calibration curve served as the readout for all following assays. The 60 mM stock solution was used to make a 120 μM target concentration of 0.2% DMSO in the appropriate buffer of the in vitro related assay (i.e. HHBS buffer). Solubilized compounds were vortex for 10 minutes followed by centrifugation for 15 minutes in 5000 RPM and the supernatant was recovered for absorbance spectra analysis between 200-400 nm.

### On Target Assays:

Two kits that enable hKv7.2/3 and hTRPV1 read-outs in a high content screen (HCS) manner were used. Electrophysiology was used to validate the readouts for selected compounds directly recording their modulations of ion-channel currents, to study the inhibition potency of our compounds on neuronal excitability, and to study the modulations of TRPV1 pH-gating.

### Kv7.2/7.3:

Cell culture: Chinese hamster ovary (CHO) cell-line cells with a constitutive expression of human Kv7.2/3 channels (B'SYS GmbH, Switzerland) were cultured in F-12 nutrient mixture (Biological Industries) supplemented with 10% fetal bovine serum (Biological Industries) and 1% penicillin-streptomycin (Biological Industries) in a humidified 5% CO₂ incubator at 37° C. To maintain the stability of human Kv7.2/7.3 expression antibiotic selection was added (Puromycin g/ml).

High content screen (HCS) using FLIPR Potassium Assay Kit: FLIPR Potassium Assay Kit (R8222 FLIPR Potassium Assay Explorer Kits, Molecular Devices) was used with the CHO/hKv7.2/3 cell line to screen molecules against the hKv7.2/7.3 ion channel target. The assay exploits the permeability of thallium ions (Tl+) through potassium (K⁺) channels. In this assay, Tl+ indicator dye is used to produce a bright fluorescent signal upon the binding to Tl+ conducted through the potassium channels. The intensity of the Tl+ signal is proportional to the number of potassium channels in the open state. Therefore, it provides a functional indication of the potassium channel activity. To activate the voltage-gate potassium channels, the cells were stimulated with a mixture of K+ and Tl+ to depolarize the cell membrane. The fluorescence increase in the assay represents the influx of Tl+ into the cell specifically through the potassium channel, providing a functional read-out of hKv7.2/3 activity using a fluorescent plate reader coupled with injectors used to activate the channels.

According to optimization process to achieve uniform and consistent screening conditions the following protocol was established: Cells were seeded in 384-well, black-walled, clear-bottomed Greiner #781091, at a density of 5000 cells per well 24 h before the assay conductance and incubated overnight in their normal growth medium. On the experiment day the medium was replaced with HBSS, HEPES, and the test compound/vehicle (0.2% DMSO). Dye solution (prepared according to the manufacturer) was added to the plates, which were incubated for 1.5 h light-protected in room temperature. Tecan Spark reader plate injectors were primed with Tl+ (1 mM Tl₂SO₄) or Tl++K+ (1 mM Tl₂SO₄; 5 mM K₂SO₄) for channel activation and data correction. Compounds effects are compared to vehicle control and NH91 or Retigabine positive control. To calculate EC50 values, the data was fitted to sigmoidal regression using Prism GraphPad. The fitting was constrained to a minimum of 1 and a maximal response of about 3.6 unless a different maximal response could be clearly identified.

### Kv7.2/7.3 Electrophysiology:

Extracellular (Bath) solution: The solution was composed of (in mM): NaCl, 140; KCl, 4; CaCl₂, 1.8; MgCl₂, 1.2; Glucose, 11; HEPES, 5.5. pH was adjusted to 7.3 with NaOH. Osmolarity was adjusted to 310 mOsm with Sucrose.

Intracellular (Pipette) solution: Pipettes were pulled from borosilicate glass (Warner Instrument Corp, USA) with a resistance of 3-7 MΩ and were filled with internal solution composed of (in mM): KCl, 130; MgCl₂, 1; K ATP, 5; EGTA, 5; HEPES, 10. pH was adjusted to 7.3 with KOH. Osmolarity was adjusted to 290 mOsm with Sucrose.

A programmable valve-linked pressurized perfusion system was used for local application of compounds nearby the cell recorded in a consistent flow rate of 2-3 ml/min. Series resistance was corrected and data were sampled at 5 kHz and low pass filtered at 2.4 kHz using MultiClamp 700B amplifier with pCLAMP11 software (Molecular Devices, USA).

To evaluate the effect of test compounds on hKv7.2/3 currents at −40 mV membrane potential (the threshold for action potential initiation) a −40 mV pulse train protocol was conducted: Membrane potential is held on −90 mV and is then clamped to −40 mV for 1.5 seconds, followed by clamping the membrane to −60 mV to obtain tail currents for 0.75 seconds, and back to the −90 mV holding potential. An interval of 30 seconds in −90 mV holding potential is kept between the sweeps.

After a steady baseline current is achieved, test compound is locally applied using the pressurized perfusion system until a maximal and stable channel modulation in −40 mV is achieved, as confirmed by recording of three similar consequent responses. Thereafter, in a similar manner, cells are perfused back to their control bath solution, to assess the reversibility of the effect of the compounds.

The current obtained at −40 mV membrane potential when test compound is injected is divided by the averaged current at −40 mV, recorded before test-compound application and following its washout, to evaluate the drug/control response.

### TRPV1:

Cell culture: Chinese hamster ovary (CHO) cell-line cells with constitutive expression of human hTRPV1 channels (B'SYS GmbH, Switzerland) are cultured in F-12 nutrient mixture (Biological Industries) supplemented with 10% fetal bovine serum (Biological Industries) and 1% penicillin-streptomycin (Biological Industries) in a humidified 5% CO2 incubator at 37° C. To maintain the stable expression of hTRPV1, antibiotic selection (G418 500 μg/ml) is applied.

Species Selection Rat, Pig, and Human DNA constructs and DNA transfection: hTRPV 1 cells were transfected with DNA constructs (Genscript). The coding regions of human (NM_080704.4), rat (NM_031982.1), and pig (XM_013981216.2) TRPV1 were inserted similarly into the multiple cloning site of pcDNA3.1(+) between the Hind III and BamH I restriction site. For CHO transfection, 1×10⁶ cells were suspended in a reaction mix (Amaxa™ 4D-Nucleofector™-LONZA) containing 5 μg of DNA for each transfection in a cuvette. The cells were transformed (Amaxa, DT-133 program for CHO transfection with p-3 Kit) and recovered for 10 minutes in RT in the hood and gently transferred with Lonza pipette dropwise into two wells of 6—wells containing prewarm medium (Ab free) to settle down. Following transfection, day before the experiment, the hTRPV1 cells were plated in a black, flat 384-well plate (Greiner #781091). The screening of molecules against CHO cells transiently transfected with rTRPV1 vs hTRPV1 vs pTRPV1 ion channel was performed using the Fluo-8 No Wash Calcium Assay Kit (Abcam, ab112129).

High content screen (HCS) using Fluo-8 No Wash Calcium flux Assay Kit: Fluo-8 No Wash Calcium Assay Kit (Abcam, ab112129) was used with CHO stably or transiently expressing TRPV1 to screen compounds against the hTRPV1 ion channel target. The cells are pre-loaded with Fluo-8AM which is membrane permeable. The AM groups of the Fluo-8AM are then being cleaved by intracellular esterase, trapping the Fluo8 in the cell. Calcium influx through activated TRPV1 channels significantly increases the fluorescence of Fluo-8. The relative fluorescence signal is calculated following background subtraction, comparing the fluorescence at each time point to its maximal level measured following Ionomycin application in the end.

According to optimization process to achieve uniform and consistent screening condition of molecules the following protocol was established: Cells were seeded in 384-well, black-walled, clear-bottomed, at a density of 5,000 cells per well 24 h before the assay conductance and incubated overnight in their normal growth medium. On the experiment day the medium replaced in each well with HBSS, HEPES, and compound/vehicle (0.2% DMSO). Dye solution (prepared according to the manufacturer) is added to the wells and plates are incubated for 1.5 h light-protected in room temperature. The Tecan Spark plate reader injectors are then primed with capsaicin and Ionomycin for channel activation and data normalization, respectively. Compounds effects are compared to vehicle control, and AMG9810 and NH91 positive controls. To calculate IC50 values, the data was fitted to sigmoidal regression line using Prism GraphPad. The fitting was constrained to a minimum of 0 and a maximal response of about 1 unless a different maximal response could be identified.

### TRPV1 Electrophysiology:

Extracellular (Bath) solution: The solution was composed of (in mM): NaCl, 140; KCl, 4; CaCl₂, 1.8; MgCl₂, 1.2; Glucose, 11; HEPES, 5.5. pH was adjusted to 7.3 with NaOH. Osmolarity was adjusted to 310 mOsm with Sucrose.

Intracellular (Pipette) solution: Pipettes were pulled from borosilicate glass (Warner Instrument Corp, USA) with a resistance of 3-7 MΩ and filled with internal solution composed of (in mM): KCl, 130; MgCl₂, 1; K ATP, 5; EGTA, 5; HEPES, 10. pH was adjusted to 7.3 with KOH. Osmolarity was adjusted to 290 mOsm with Sucrose. A programmable valve-linked pressurized perfusion system was used to locally apply compounds nearby the cell recorded, in a consistent flow rate of 2-3 ml/min. Data were sampled at 5 kHz and low pass filtered at 2.4 kHz using MultiClamp 700B amplifier with pCLAMP11 software (Molecular Devices, USA).

Membrane potential was held in −60 mV. To measure the inhibitory effects of our test compounds on hTRPV1 activity, hTRPV1 gating was achieved by fast short applications of hTRPV1 activator Capsaicin (100 nM, 6 s), with or without compound (>3 minutes) co-application. A 3-minute time interval was set between sequential Capsaicin injections to allow its washout and cell recovery. All compounds are injected in the vicinity of the cells using the pressurized perfusion system.

The drug current response, when capsaicin (co-applied with test compound) is injected following >3 minutes of pre-incubation with the test compound, is divided by the averaged control currents, obtained when capsaicin is injection alone, before compound incubation and following its washout, to evaluate the drug/control response.

For the study of TRPV1 pH-activation, the following pH 5.5 MES based extracellular activation solution is used:

Extracellular (bath) pH 5.5 activation solution: The solution was composed of (in mM): NaCl, 140; KCl, 4; CaCl₂, 1.8; MgCl₂, 1.2; Glucose, 11; MES, 5.5. pH was adjusted to 5.5. Osmolarity was adjusted to 310 mOsm with Sucrose.

### Human NPCs:

Cell culture and differentiation: Human PSC-derived neural progenitor cells (Stem Cell Catalog No. 70901 and 70902) were cultured and expanded on Matrigel coated 6-wells plates, using neuronal progenitor medium containing: Neurobasal media supplemented by non-essential amino-acids, 1%; Glutamax, 1%; B27, 2%, FGF2, 20 ng/ml). hNPC were then plated onto PDL and Matrigel coated 12 mm coverglass at a density of about 50,000 cells/well of a 6 well, in DMEM/F12; 10% KSR; 1% P/S and A83-01 (2 M) from days 0-5. From days 0-9 the medium contained also CHIR99021 (6 μM). From Day 3-9 the medium included R04929097 (2 μM) and SU5402 (3 μM). From day 9 and onwards, the media change (Neurobasal Media contains: NT-3; BDNF; NGF; GDNF) was performed every other day by replacing only 50% of the media after CO₂ equilibrating. On day 12 cells were incubated with Mitomycin C chemotherapeutic agent (2.5 μg/ml, 2 hours, 37° C.) to avoid glial cells proliferation.

Electrophysiology: To characterize the neuronal properties with electrophysiology recordings, round small neurons (20-40 μm in diameter) were selected from day 31 and onwards. Cells were monitored for their polarized negative membrane potential and for their ability to evoke consistent spikes train responses to repeated positive current injections as a mark of achieving electrical maturation before testing the effect of the tested compounds on neuronal excitability. Membrane excitability was monitored using current clamp. Positive current steps of different amplitude were injected (400 ms) to induce spikes trains before and following compound application.

### RatDRGs:

rDRGs isolation and primary cell culture: Briefly, rat DRGs from all spinal levels were carefully removed and collected in HBSS on ices, connective tissue of the epineurium surrounding the ganglion was removed and cells were dissociated using Collagenase-II and trypsin dissociation solutions. DRGs were dissociated further, passed through glass Pasteur pipettes to obtain single cells. Then dissociation solution was changed to 5% FBS containing DRG neuronal culture medium and plated onto ECL coated 12 mm cover glass.

Additional rDRGs experiments were performed using Lonza primary neonatal rat (Sprague Dawley neonatal; P2,3) DRG neurons (R-DRG-505, Lonza) plated and cultured according to the manufacturer.

These cryopreserved DRG cells were prepared from freshly isolated and dissociated spinal cord dorsal root ganglia and comprise a normal distribution of neurons and glia (schwann cells).

Electrophysiology: Cells were used for electrophysiological recording in the 48 hours following rDRG isolation. Round small neurons (20-40 m in diameter) were selected for electrophysiology recordings, to select for small nociceptive neurons that propagate pain sensation in vivo and that are expressing both Kv7.2/3 and TRPV1 targets. Membrane excitability was monitored using current clamp. Positive current steps of different amplitude were injected (400 ms) to induce spikes trains before and following compound application.

### Off-Target Assays:

### Kv7.3/5, Kv7.4 Assay:

Cell culture: Chinese hamster ovary (CHO) cell-line cells were cultured in F-12 nutrient mixture (Biological Industries) supplemented with 10% fetal bovine serum with 1% penicillin-streptomycin (Biological Industries) in a humidified 5% CO₂ incubator at 37° C. To express the appropriate gene either Kv7.3/5, Kv7.4 or TRPA1 5 μg plasmid was transfected using Amaxa, DT-133 program, with p-3 Kit.

FLIPR Potassium Assay Kit: The FLIPR Potassium Assay Kit (R8222 FLIPR Potassium Assay Explorer Kits, Molecular Devices) was used with transfected CHO cells.

According to optimization process, to achieve a uniform and consistent screening condition of molecules, the following protocol was established: 2 days following transfection cells were seeded in 384-well, black-walled, clear-bottomed, at a density of 5,000 cells per well 24 hours before the assay conductance and incubated overnight in their normal growth medium. On the experiment day the medium was replaced with HBSS, HEPES, and the tested compound/vehicle (0.2% DMSO). Dye solution (prepared according to the manufacturer) was added to the wells and plates were incubated for 1.5 hour light-protected in room temperature. The Tecan Spark plate-reader injectors were primed with Tl+ (Tl₂SO₄) or Tl++K+ (Tl₂SO₄; K₂SO₄) for channel activation and data correction. Compounds effects were compared to vehicle control and Retigabine positive control (Known IC50=1.5-5 μM). To calculate IC50 values, the data was fitted to sigmoidal regression using Prism GraphPad.

### Trpa1 Assay:

Cell culture: Chinese hamster ovary (CHO) cell-line cells were cultured in F-12 nutrient mixture (Biological Industries) supplemented with 10% fetal bovine serum with 1% penicillin-streptomycin (Biological Industries) in a humidified 5% CO₂ incubator at 37° C. To express the appropriate TRPA1 5 μg plasmid was transfected using Amaxa, DT-133 program, with p-3 Kit.

High content screen (HCS) using Fluo-8 No Wash Calcium flux Assay Kit: Fluo-8 No Wash Calcium Assay Kit (Abcam, ab112129) was used with CHO transiently transfected TRPA1 to screen compounds against the hTRPA1 ion channel target. The cells are pre-loaded with Fluo-8AM which is membrane permeable. The AM groups of the Fluo-8AM are then being cleaved by intracellular esterase, trapping the Fluo8 in the cell. Calcium influx through activated TRPA1 channels significantly increases the fluorescence of Fluo-8. The relative fluorescence signal is calculated following background subtraction, comparing the fluorescence at each time point to its maximal level measured following Ionomycin application in the end.

According to optimization process to achieve uniform and consistent screening condition of molecules the following protocol was established: Cells were seeded 2 days following transfection in 384-well, black-walled, clear-bottomed, at a density of 5,000 cells per well 24 h before the assay conductance and incubated overnight in their normal growth medium. On the experiment day the medium replaced in each well with HBSS, HEPES, and compound/vehicle (0.2% DMSO). Dye solution (prepared according to the manufacturer) is added to the wells and plates are incubated for 1.5 h light-protected in room temperature. The Tecan Spark plate reader injectors are then primed with AITC and Ionomycin for channel activation and data normalization, respectively. Compounds effects are compared to vehicle control, and known inhibitor of TRPA1-A967079. To calculate IC50 values, the data was fitted to sigmoidal regression line using Prism GraphPad. The fitting was constrained to a minimum of 0 and a maximal response of about 1 unless a different maximal response could be identified.

### Herg Assay:

Cell culture: Chinese hamster ovary (CHO) cell-line cells with a constitutive expression of human hERG channels (B'SYS GmbH, Switzerland) were cultured in F-12 nutrient mixture (Biological Industries) supplemented with 10% fetal bovine serum (Biological Industries) and 1% penicillin-streptomycin (Biological Industries) in a humidified 5% CO₂ incubator at 37° C. To maintain the hERG expression stability of the CHO cell line, antibiotics selection (G418 200 μg/ml and Hygromycin 500) was applied.

FLIPR Potassium Assay Kit: The FLIPR Potassium Assay Kit (R8222 FLIPR Potassium Assay Explorer Kits, Molecular Devices) was used with CHO/hERG cell line, for screening compounds against the hERG ion-channel off-target, as detailed hereinabove.

According to optimization process, to achieve a uniform and consistent screening condition of molecules, the following protocol was established: Cells were seeded in 384-well, black-walled, clear-bottomed, at a density of 7,500 cells per well 24 hours before the assay conductance and incubated overnight in their normal growth medium. On the experiment day the medium was replaced with HBSS, HEPES, and the tested compound/vehicle (0.2% DMSO). Dye solution (prepared according to the manufacturer) was added to the wells and plates were incubated for 1.5 hour light-protected in room temperature. The Tecan Spark plate-reader injectors were primed with Tl+ (1 mM Tl₂SO₄) or Tl++K+(1 mM Tl₂SO₄; 5 mM K₂SO₄) for channel activation and data correction. Compounds effects were compared to vehicle control and Terfenadine positive control (Known IC50=200 nM). To calculate IC50 values, the data was fitted to sigmoidal regression using Prism GraphPad.

### Hepg2 Assay:

Cell culture: HepG2 cell-line cells were cultured in EMEM (ATCC 30-2033) medium supplemented with 10% fetal bovine serum (Biological Industries) in a humidified 5% CO₂ incubator at 37° C.

ATPlite™ 1step Kit: ATPlite™ 1step kit (PerkinElmer) was used in a 384-wells format to assess potential liver toxicity using HepG2 cells. This is an ATP detection system based on firefly luciferase luminescence for the quantitative evaluation of proliferation and cytotoxicity of cells. ATP is a marker for cell viability because it is present in all metabolically active cells and its concentration declines very rapidly when the cells undergo necrosis or apoptosis.

According to an optimization process done to achieve uniform and consistent screening condition of molecules, the following protocol was established: Cells were seeded in 384-well, black-walled, clear-bottomed, at a density of 4000 cells per well 48 hours before the assay and incubated overnight in their normal growth medium. On day two (48 hours before viability measurement), the tested compounds and the positive control were applied to the wells according to the plate design. Compounds were dissolved in phenol-free growth medium at 0.1% DMSO concentration and their concentrations were identified using a solubility assay as described herein. 48 hours later, reconstituted lyophilized substrate solution was added to each well and the sealed 384-well microplate was mixed for 2 minutes at 1100 rpm in an orbital microplate and plates were incubated for 10 minutes in the dark at room temperature. Right after, luminescence was measured in the Tecan plate reader. To estimate the LD50 and LD25 values the data was fitted to sigmoidal regression using Prism-GraphPad.

### Druggability:

### Metabolic Stability and SAR Driven Optimization of Human Liver Microsome (HLM) Values:

Metabolic stability refers to a compound susceptibility to biotransformation. Both in vitro half-life (t_{1/2}) and intrinsic clearance (CLint) are typically utilized to express metabolic stability. CLint represents the maximum activity of liver microsomal proteins/hepatocytes towards a compound, without involving other physiological parameters such as hepatic blood flow and drug binding within the blood matrix. Since oral drugs first pass metabolism is via the liver the CLint parameter is an important factor.

Liver microsomes which are subcellular particles derived from endoplasmic reticulum of hepatic cells were used for evaluating the tested compounds. These particles are rich in drug metabolizing enzymes, including the cytochrome p-450 family. Liver microsomes are a recommended test system for various in vitro drug metabolism and pharmacokinetics (DMPK) studies. CLint values lower than 100 l/min/mg are considered suitable for drug development.

### Logd Measurements:

LogD, a Log of partition of a chemical compound between a lipid phase and an aqueous phase, was measured at pH 7.4 (which equals LogP), in order to calculate accurately the LLE and to uncover compounds that exhibit improved physiochemical properties.

### Lle Measurements:

Ligand-lipophilicity efficiency (LLE) is a parameter used in drug design and drug discovery to evaluate the quality of research compounds, linking potency and lipophilicity.

To follow LLE values, the following equation was used:

LLE=−Log(IC50)−c Log P

Suitable LLE value are typically higher than 3 or higher than 5.

### Example 1

### Structure Activity Relationship (SAR) Studies

As discussed hereinabove, one of the most potent compounds disclosed in WO 2019/073471 is referred to therein as NH91.

NH91 features the following biological and physicochemical parameters:
TRPV1 IC50: 8 μM
Kv 7.2/7.3 EC50: 0.7 μM
CLogP: 5.3
LLE TRPV1: −0.35
LLE Kv 7.2/7.3: 0.8
Solubility: 20 μM

With the aim at improving the pharmaceutical performance of NH91, four pharmacophoric sites were marked for possible modifications: Ring A, the bridging amine, Ring B and the side chain (meta to the amine bridge), as shown in Scheme 1 below.

A preliminary library of several dozens of compounds was synthesized and biological measurements and calculations (TRPV1 IC50, Kv7.2/7.3 EC50, LLE, Kinetic solubility, etc.) were performed as described herein.

The preliminary library included the following modifications:

Ring A modifications: replacing the isopropyl group by other groups, such as tert-butyl, cyclopentane, pyridine, etc.; or replacing one of the chloro substituents by e.g., other halo substituents, electron-withdrawing substituents, etc.

Bridge modifications: replacing the amine bridge by sulfonamide or an ether moiety (—O—).

Ring B modifications: replacing the phenyl by pyridine.

Side chain modifications: replacing the linear hydroxyalkyl by a hydroxy-substituted cycloalkyl or heteroalicyclic, or by a branched hydroxyalkyl.

The data obtained for the compounds of this preliminary library (not shown) indicated that:
(i) Introducing bulky hydrophobic substituents on Ring A, such as tert-butyl and cycloalkyls, improved activity on both channels but no improvement was observed in LLE
(ii) Introducing bulky hydrophilic substituents on Ring A, improved LLE but resulted in reduced activity
(iii) Replacing the amine bridge by sulfonamide resulted in substantial drop in the potassium channel opening
(iv) Replacing the amine bridge by an ether moiety resulted in improved parameters but reduced solubility
(v) Replacing Ring B by pyridine resulted in reasonable parameters
(vi) Out of the tested structures, 35 were side chain modifications, since previous studies have shown that both TRPV1 and Kv7.2/7.3 are sensitive to synthetic modifications at this portion of the molecule. The obtained data supported a desired effect of these modifications on the LLE values, along with retained activities and lower cLogP values.

Based on the data obtained for the preliminary library of compounds, additional libraries, including additional modifications in one or two of the pharmacophoric sites shown in Scheme 1, were designed, synthesized and tested. Additional designs mainly included replacing the phenyl in Ring A and/or Ring B by a heteroaryl such as pyridine, imidazole or a heteroalicyclic; replacing the amide bridge by —S—, —S(═O)—, —P(OH)—, —CH(OH)—, or —C(═O)—; replacing the Ring A-NH-aniline moiety by a rigid heteroaromatic moiety such as indole; and further modifications of the side chain site.

Based on the data obtained for the additional libraries (not shown), three main chemotypes were shown to exhibit desired properties in terms of dual activity on TRPV1 and the potassium channel, solubility, LLE and stability.

These chemotypes include modifications of the bridge as follows:
Chemotype 1: replacing the Ring A-NH—aniline moiety by a rigid indole moiety; see, FIG. 1 for representative structures
Chemotype 2: replacing the amine bridge by —S— or —O—; see, FIG. 2 for representative structures
Chemotype 3: replacing the amine bridge by —CH(OH)—; see, FIGS. 3A-B for representative structures
Chemotype 4: replacing the amine bridge by —C(═O)—; see, FIGS. 3A-B for representative structures

In addition, amide isosteres have been considered as replacing the amide moiety within the side chain portion. Exemplary such isosteres include an oxetane structure (see, for example, FIG. 1; compound 000526) and triazole structure (see, for example, FIG. 1; compound 000527).

Further in addition, compounds featuring a heteroaryl (e.g., pyridine) as Ring B were considered. Exemplary such compounds of chemotype 1 include compounds 000661, 000662, 000663 and 000649 (see, FIG. 1).

The biological and physicochemical properties of representative compounds of each chemotype are presented in Table 1 below.

It is to be noted that the chemotype 3 compounds (featuring a dihydroxy structure) and corresponding chemotype 4 compounds (featuring a ketone bridge) were found to be conceptually interesting, since the dihydroxy chemotype 3 can be oxidized into its corresponding ketone structure by the liver enzyme Alcohol dehydrogenase (ADH), as shown in FIG. 3B. As the enzymatic reaction is reversible, the two structures could be possibly generated according to the body's needs. Under this scenario, dihydroxy chemotype 3 is expected to be active mainly against Kv7.2/7.3 (EC50=5.6 μM) whereas its correlated in-situ oxidized ketone structure chemotype 4 is expected to be active mainly against TRPV1 (IC50=8 μM).

TABLE 1

| | TRPV1 | Kv7.2/7.3 | | | | | | |
|---|---|---|---|---|---|---|---|---|
| | IC50 | IC50 | HLM Clint | Kinetic | | hERG IC50 | | CNS |
| Compound | [μM] | [μM] | [μl/min/mg] | Sol. | LogD | [μM] | | PMO |
| 421-6 | 2.1 | 1.1 | 55.5 | 3.4 | 5.47 | >>2.5 | (0%) | 3.1 |
| 414 | 1.1 | 1.8 | 686 | | | >>2.5 | (0%) | 2.6 |
| 415 | 7.7 | 2.2 | 839 | 26.7 | 4.25 | >>23 | (0%) | |
| 417 | 26.6 | 5.6 | 89.1 | | | >>2.5 | (0%) | 2.8 |
| 541 | 3.2 | 4.7 | 772.2 | 2 | 4.78 | | | |
| 552 | 1.0 | 12 | 27.2 | 5 | 4.77 | | | |
| 627 | 1.9 | 0.54 | <9.6 | 6 | 4.85 | >>2.5 | (0%) | 3.89 |
| 533 | 21.2 | 1.5 | 657.9 | 41 | 4.36 | >>50 | (0%) | |
| racemic | | | | | | | | |
| 417-4 | 8 | 13.4 | 91.84 | 66 | 4.08 | >>35 | (0%) | |
| 569 | 10 | 3 | 120 | 27 | 4.47 | | | |
| 573 | 3 | 7 | 441 | 24 | 4.34 | | | |
| 649 | 3.3 | 5.2 | 27.3 | 10 | 4.19 | | | |
| 661 | 19.3 | 1.1 | 72 | 40 | 3.55 | >>16.6 | (0%) | 4.0 |
| 662 | agonist | 8.0 | 38.1 | 6 | 3.49 | | | |
| 663 | 2.2 | 5.9 | 53.6 | 26 | 3.52 | | | |

### Example 2

### Computational Modeling

Molecular docking and quantitative Structure Activity Relationship modeling (qSAR) represent the major computational tools employed in computational chemistry. Molecular docking applies the target protein's 3D structure to locate various molecules in a preferred active conformation inside a chosen virtual binding pocket (i.e., Structure Based Drugs Design). qSAR applies various physicochemical as well as 2D and 3D structural elements in a chosen set of 10 molecules to predict measured parameters of compounds (see, Sharmna, S., Recent trends in QSAR in modelling of drug-protein and protein-protein interactions. Comb. Chem. High Throughput Screen. 2020).

### Molecular Docking:

To perform docking experiments, homology model of the human TRPV 1 was generated. For that purpose, the following steps were performed:
1. Building multiple homology models: one using 5IRX (Prati, F. Stem Cell Translational medicine, 2017; 6:369-381), ligand-bound structure, and 3J5P (SA CAI, et al. Stem Cell Translational medicine, 2017; 6:369-381), the apo structure (“closed conformation”).
2. Analyzing the pockets on both and comparing to mutation data.
3. Docking identified lead compounds to both homology models.

A homology model which is based on the published 3J5P model (SA CAI et al. 2017 supra) was thereby generated (not shown). This model showed additional plausible binding modes and was consistent with the rat/human difference at residue 547.

Homology model of the human Kv7.2/7.3 was based on the open state of Kv7.1 (Peretz et al., Proceedings of the National Academy of Sciences August 2010, 107 (35) 15637-15642).

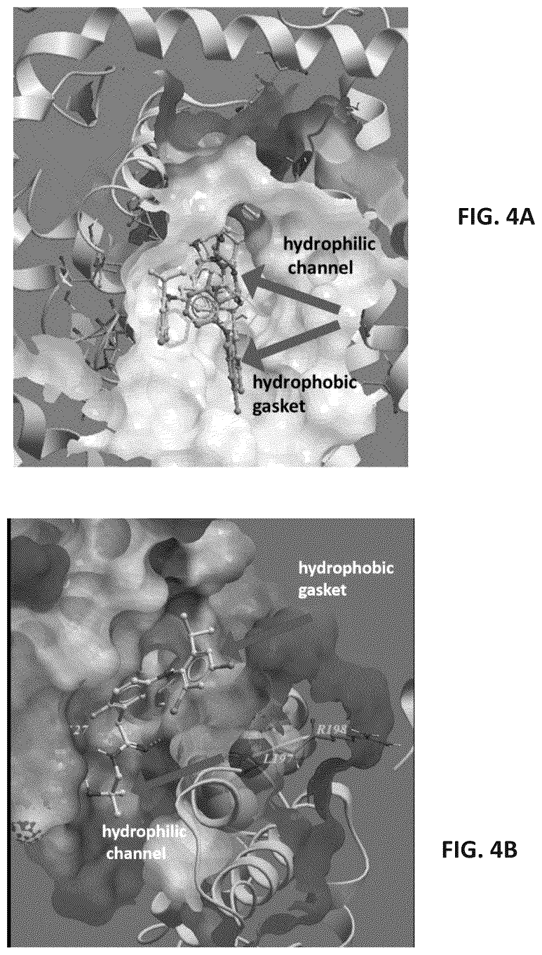

FIG. 4A presents the docking of NH91 (000091; bright yellow), of the known TRPV1 inhibitor resiniferatoxin (RTX), and of Compound 228 (a compound as described, for example, in WO 2004/035037) inside the vanilloid pocket of the respective homology model. FIG. 4B presents the docking of NH91 inside the vanilloid pocket of the homology model of the Kv7.2/7.3.

As can be seen, the hydrophobic gaskets and hydrophilic channels are shared structural motifs of both proteins, which allow the design of dual Kv7.2/7.3 and TRPV1 modulators.

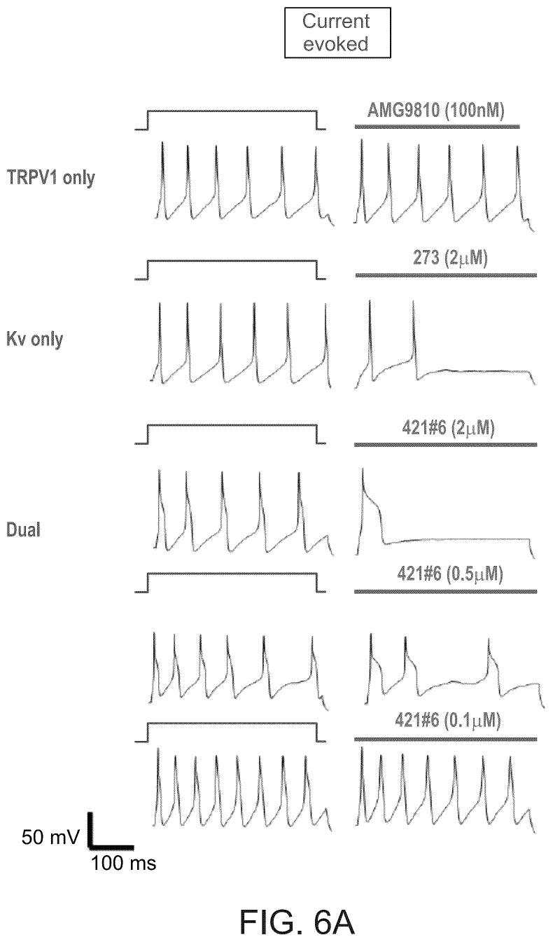

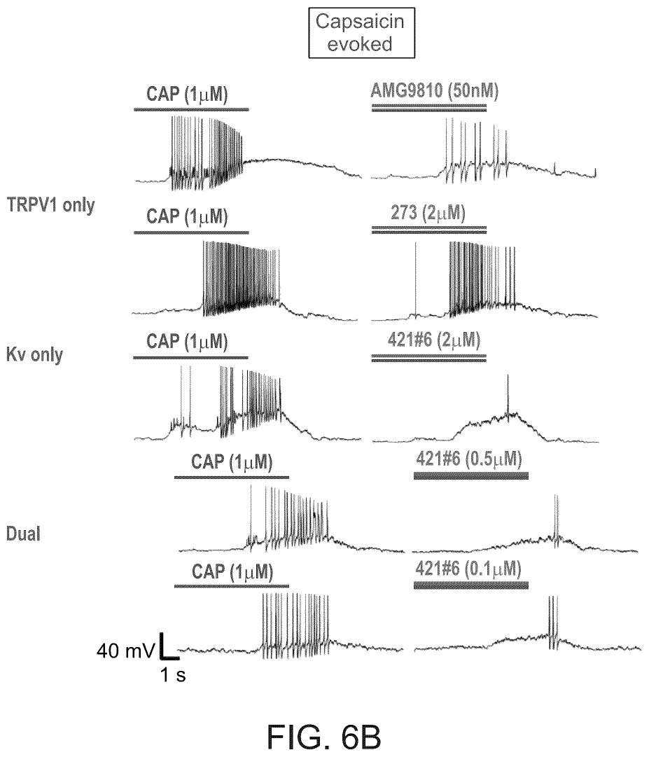

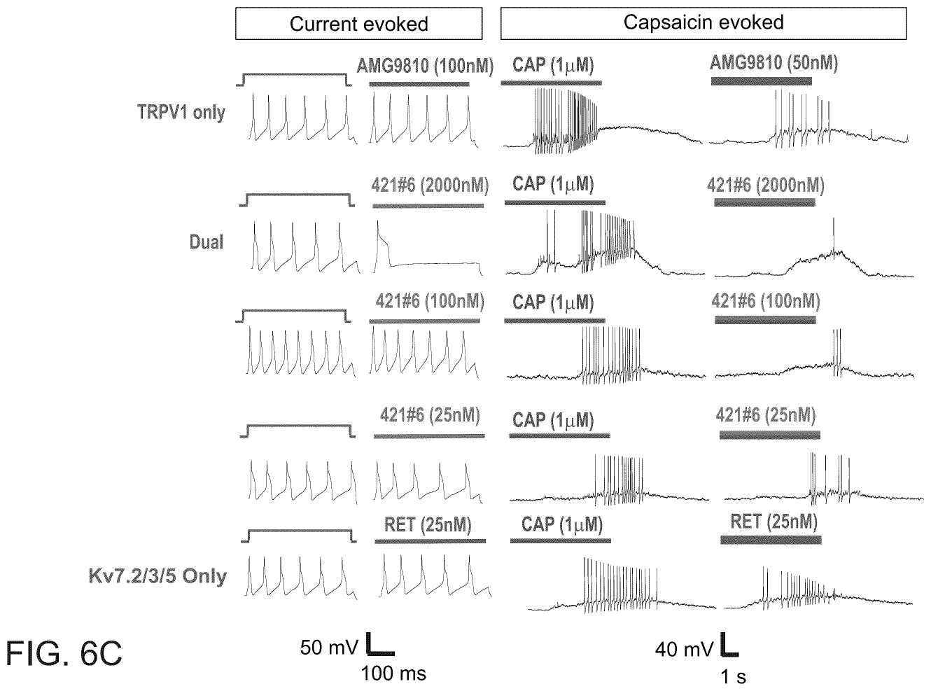

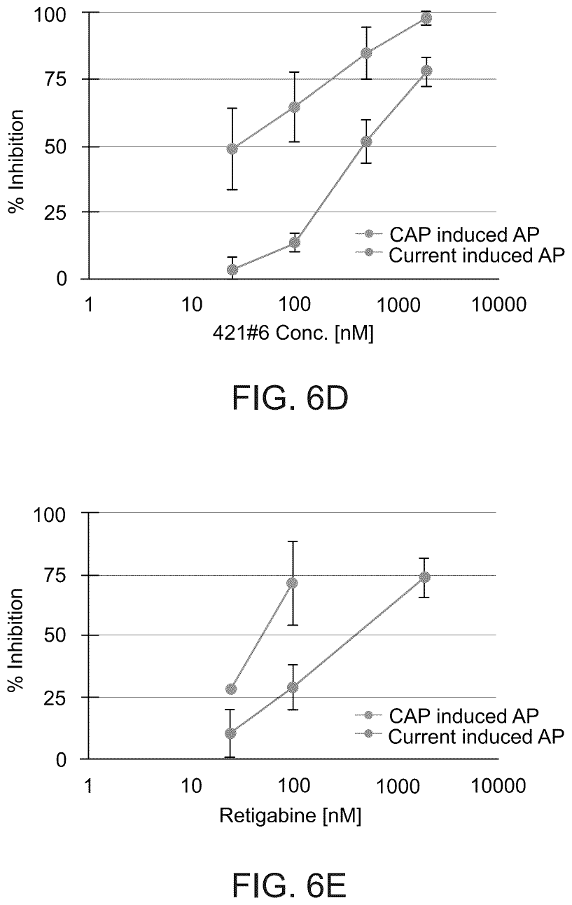

Based on the three-dimensional structure of the human TRPV1 and the uncovered preferred binding poses for the tested compounds (FIG. 6A), the following was deduced:
=The newly designed compounds have high probability of binding and interacting with the TRPV1's vanilloid pocket (i.e., capsaicin binding pocket);
=The ‘A’ and ‘B’ rings preferably interact with a hydrophobic surface area (i.e., hydrophobic gasket);
=The polar side chain is preferably located into a hydrophilic channel.

Based on the three-dimensional structure of the human Kv7.2/7.3 and the uncovered preferred binding poses for the tested compounds (FIG. 4B), the following was deduced:
=The terminal hydroxyl (—OH) as well as the hydrophobic A ring preferably interact with a hydrophilic channel and a hydrophobic gasket, respectively;
=The aniline bridge results in a non-optimized rigid structure, pointing towards modifications at this pharmacophoric site as a promising pathway for improved activity against Kv7.2/7.3.
   Quantitative Structure Activity Relationship Modeling (qSAR):

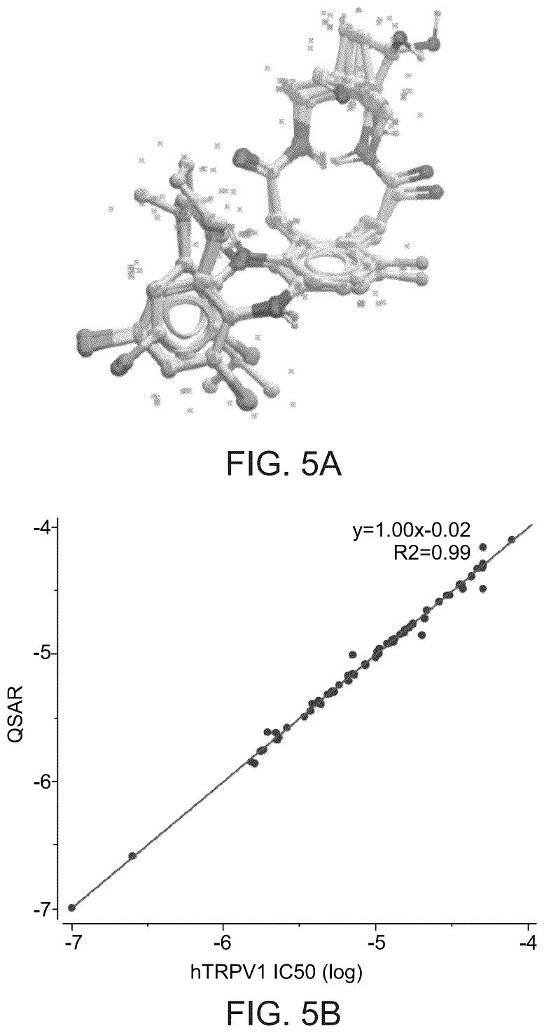

For creating such models, some of the hit compounds described herein were superimposed one on top of the other in their calculated 3D stable conformation and a pharmacophoric representation was created, as shown in FIG. 5A. Based on the pharmacophoric representation, various learning models were created, which allowed correlating all the IC50/EC50 values (generated for TRPV1 or Kv7.2/7.3, respectively) with their structural elements, as shown in FIG. 5B for TRPV1.

Exemplary data of representative TRPV1 qSAR modelling outcome is presented in Table 2.

TABLE 2

| Compound No. | qSAR IC50 [μm] | Experimental IC50 [μm] |
|---|---|---|
| 414 | 6.3 | 1.1 |
| 415 | 7.8 | 7.8 |
| 480 | 2.1 | 1.4 |
| 422 | 8.3 | 2.2 |
| 405 | 88 | 125 |

The capability of a representative TRPV1 qSAR model to highly correlate between the experimental IC50 values and qSAR predictions is clearly shown.

A computational infrastructure was therefore generated, consisting of both homology models and docking capabilities as well as qSAR learning models.

### Example 3

### Efficacy Studies

### Effect on Rat DRG Neurons:

The inhibitory potency of the newly designed compounds was studied on neuronal excitability in an experimental system of high physiological relevance, primary neonatal rat DRG neurons (Sprague Dawley neonatal; P2,3; R-DRG-505, Lonza). These cryopreserved DRG cells are prepared from freshly isolated and dissociated spinal cord dorsal root ganglia and comprise a normal distribution of neurons and glia (schwann cells).

The inhibitory potency was tested both on current-induced and Capsaicin-induced neuronal activity. The obtained data is shown in FIGS. 6A-E.

As can be seen in FIG. 6A, with current stimulation, the inhibitory potency of the tested compounds is mediated and identified through their effects on the voltage-activated effectors, such as the Kv7.2/3 target. As can be seen in FIG. 6B, Capsaicin evoked responses identify the contribution of both the Capsaicin-gated TRPV1 target, which depolarize the membrane upon activation, which consequently activates the voltage-gated Kv7.2/3 target downstream. Thus, with both targets being activated, the add-on effect of dual targeting of TRPV1 and Kv7.2/3 can be compared with only single Kv7.2/3 targeting compound such as Retigabine that have similar EC50 for Kv7.2/3 as 421-6.

More specifically, AMG9810 inhibitory potency was tested first, both on current and Capsaicin evoked neuronal activation. Capsaicin evoked firing was reduced by about 50% with AMG9810 (50 nM) application. On the contrary and as expected, the current evoked response remains unchanged since AMG9810 does not target any known voltage dependent component of the action potentials firing.

Next, compound 273, showing Kv-only targeting (Kv7.2/3 EC50=1.3 μM), was examined (2 M) and showed a partial reduction of current evoked neuronal activation. Capsaicin evoked neuronal activation, which reads-out the combination of both targets, also has showed a similar reduction in its AP response, supporting the effect of Kv activation as a general mechanism of reduction in action potential firing regardless of the activation mechanism (current or Capsaicin induction).

Then, compound 421-6, which exhibits TRPV1 and Kv7.2/3 dual targeting, was examined (Kv7.2/3 EC50=2.1 μM; TRPV1 IC50=1.8 μM). Applying 2 μM of 421-6, around both its Kv7.2/3 EC50 and TRPV1 IC50, dramatically reduced the AP response to both current and Capsaicin activation, showing inhibitions of 77.7±5.6% (N=3) and 97.9±2.1% (N=2) respectively. The pronounced inhibition of neuronal voltage dependent AP activity was higher than the 50% inhibition of Kv7.2/3 activity observed measuring the K influx through these channels expressed in heterologous system. This might reflect changes evolving from aspects resembling the different nature of these two readouts, given that one directly measures the gradually accumulated ion conducted through the gated ion-channel target and the other reads the physiological relevant outcome which is a threshold all-or-none phenomenon regulated also by the target. Possible different target interactions and compositions (e.g. different Kv subunits stoichiometry and relative expression) in the endogenous versus the heterologous system may also explain these findings.

Since neuronal excitability is a physiological predictive readout of pain propagation and in light by the apparent high potency of compound 421-6 in reducing neuronal firing, its IC50 value inhibiting firing responses in DRGs neurons was tested. To this end, the activity of compound 421-6 was tested at various nM concentrations.

The obtained data, along with comparative data obtained for retigabine (RET), is shown in FIGS. 6A-E. Diluting 421-6 significantly reduced its inhibition of current induced AP responses, reaching its IC50 around 500 nM (51.2±8.4% N=3), retaining a significantly higher inhibition of the capsaicin induced AP response, which reflects the impact of dual action on both targets (86.5±8.4%, N=4). Furthermore, 421-6 dilution to a concentration of 100 nM, inhibited current induced AP activity by only 10.1±3.4% N=2, while still preserving a high inhibition potency of capsaicin induced AP response (63.4±17.8%, N=3), emphasizing both the high potency and synergistic activity of this dual-targeting compound.

Compound 421-6 dual targeting compound displays a <100 nM inhibition of capsaicin induced neuronal firing. This high inhibition potency, significantly above the sum of inhibitions contributed by each target alone, indicates the synergistic effect of 421-6. This synergism evolves (1) the high inhibition potency for such a compound (2) a superior specificity that emerges from a higher activity occurs only where both targets are co-expressed and their signaling pathways are crossing, which is unique to the nociceptive sensory neurons. Such a synergism might occur when a second messenger of one target is a modulator of the other one. For example, TRPV1 inhibition by the compounds of the present embodiments inhibits the Ca2+ influx through this channel which consequently might relief calcium mediated inhibition of Kv7.2/3 channel, to synergistically activate the M-current together with the direct Kv7.2/3 activation by the same compound.

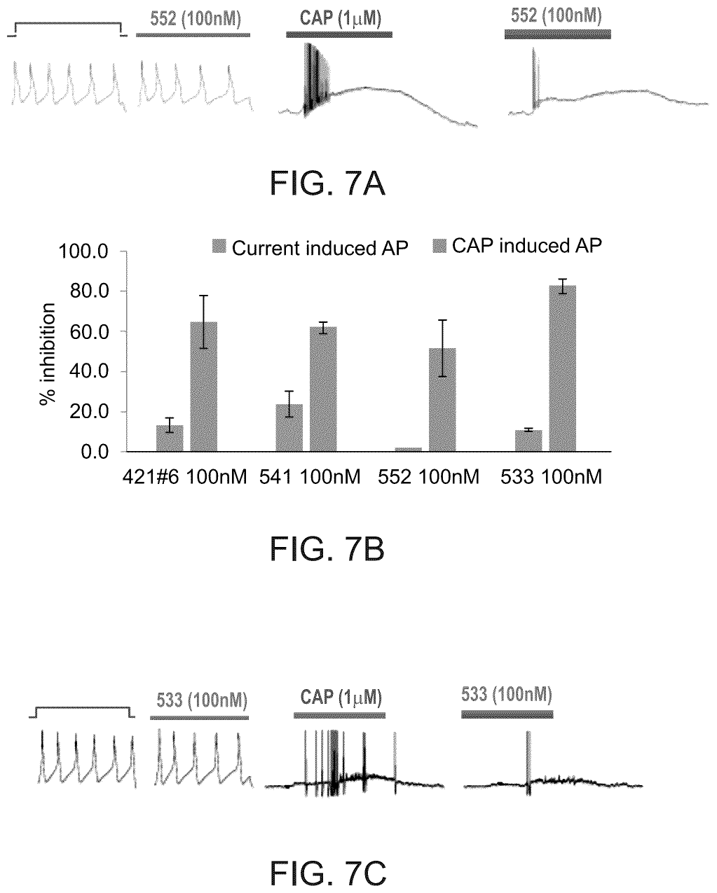

FIGS. 7A-C present data obtained in these assays for other representative compounds according to the present embodiments, denoted Compounds 552 and 541 (see, FIG. 1) and 533 (see, FIG. 3B).

### Neuropathic Model in Rat DRGs:

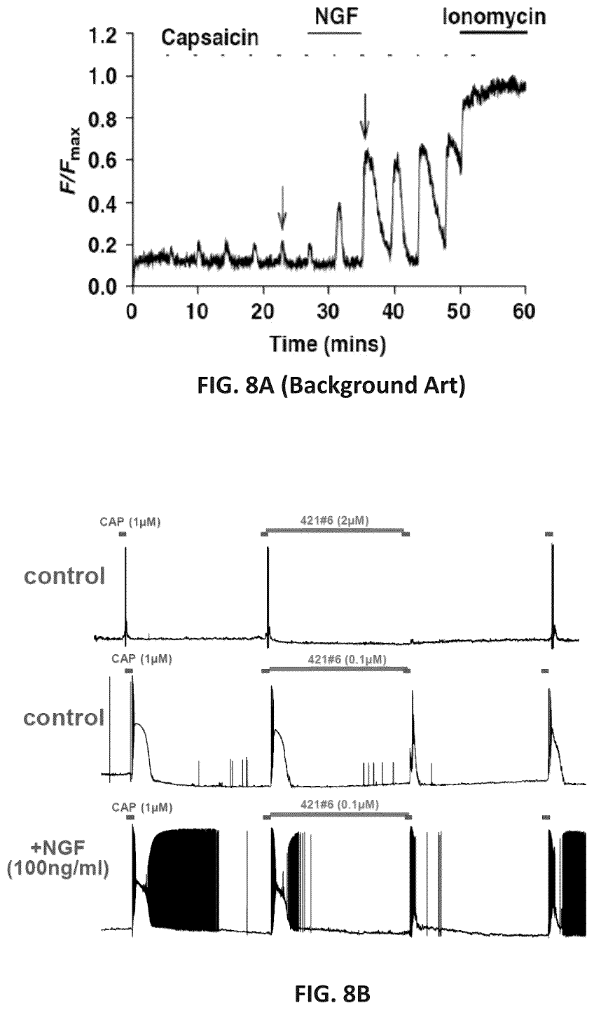

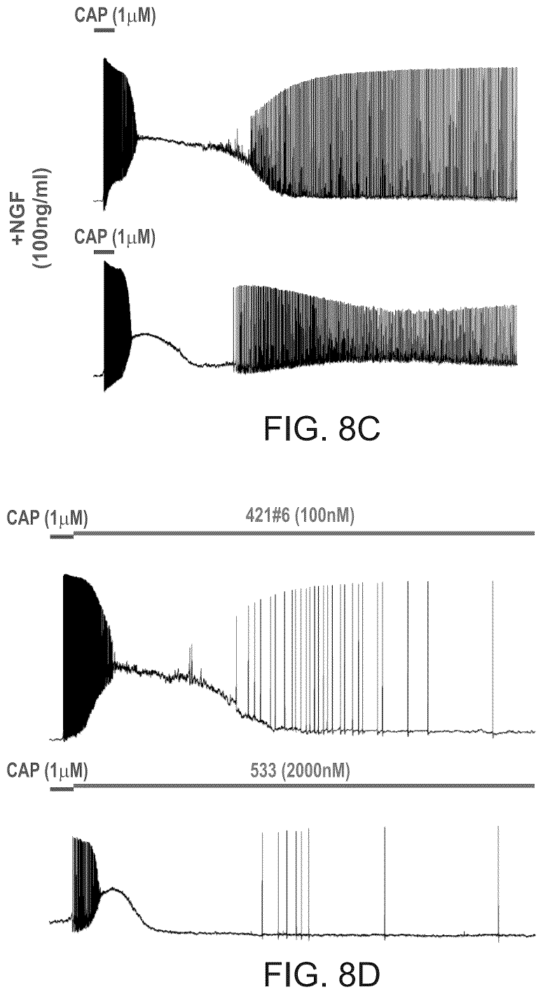

DRGs treated with NGF show tetanic action potential burst in response to CAP application, mimicking neuropathic like response. See, Background Art FIG. 8A. NGF enhances TRPV1 function using Calcium-dependent fluorescence, F, relative to maximal fluorescence, Fmax, as a function of time from a single HEK293 cell stably transfected with TrkA and transiently transfected with hTRPV1. Pulses of capsaicin (100 nM, applied as shown at top) elicited submaximal increases in [Ca]i. Exposure to NGF (100 ng/ml, see top) enhanced the capsaicin induced calcium increase. Arrows show responses used for calculation of sensitization ratio.

As shown in FIGS. 8B-D, compound 421-6 alleviates the capsaicin induced tetanic action potential burst at levels below EC50, supporting the advantage of dual Kv/TRPV1 modulation. As shown in FIG. 8B, the second tetanic burst is modulated as 421-6 reaches steady state concentration in the recording chamber. The effect is maintained following the third application of capsaicin as the concentration decreased with the washout, suggesting a slow off rate kinetics. As can be seen following the fourth capsaicin application, the effect is reversible.

### Effect on Human NPCs:

To study the inhibitory potency of exemplary leading compounds on neuronal excitability in an experimental system that resembles their effect on human pathophysiology, human neural progenitor cells (hNPCs) were differentiated to human sensory neurons and following their electrical maturation, their excitability was examined with or without the tested candidates.

Differentiated human sensory neurons went through electrical maturation, showing polarized resting membrane potential that became more and more negative along maturation, as expected from mature neurons with their increased population of K channels, shifting the resting membrane potential toward the potassium reversal potential.

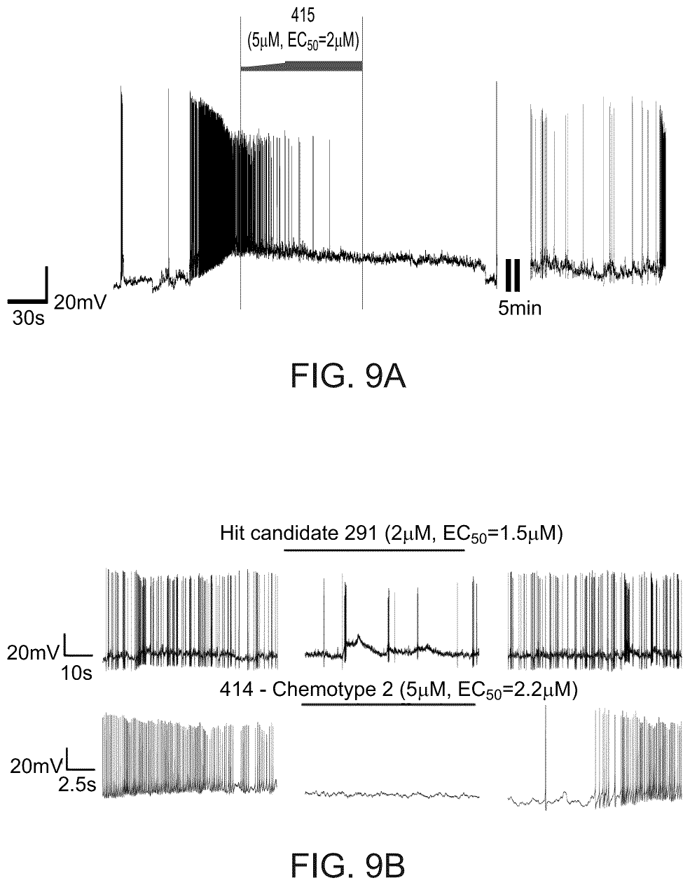

The inhibitory potency of the newly designed compounds as described herein was evaluated based on their ability to reduce the spontaneous neuronal activity. As shown in FIG. 9A, a differentiated human sensory neuron, shows spontaneous activity which was significantly reduced following application of a chemotype 2 compound 415 (5 μM) in a reversible manner. Compound 415 displayed a strong inhibitory potency in human sensory neurons, inhibiting spontaneous firing and the need to approach the targets at a much lower, predicted nM concentrations for effective concentration without potential adverse effects.

FIG. 9B presents the data obtained for a chemotype 2 compound 414, compared to compound 219, which bears an aniline bridge:

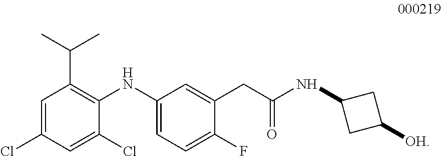

These data show the strong inhibition potency of the newly designed compounds as described herein on the excitability of rat and human sensory neurons.

### Example 4

### Further Insights on the Mechanism of Action

### Effect of Racemic Mixture and Separated Enantiomers:

To test whether different enantiomers of molecules that feature a single chiral carbon exhibit different activity, the compound 533 was selected as a representative example (* denotes a chiral center).

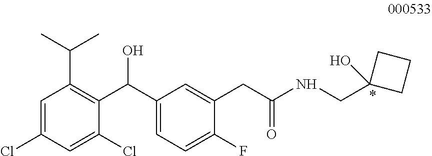 [SMILES: CC(C)c1cc(Cl)cc(Cl)c1C(O)c1ccc(F)c(CC(=O)NCC2(O)CCC2)c1]

The racemic mixture and the two enantiomers, separated using SFC methodology and denoted as 533p1 and 533p2, were tested in efficacy studies as described in Example 3 hereinabove.

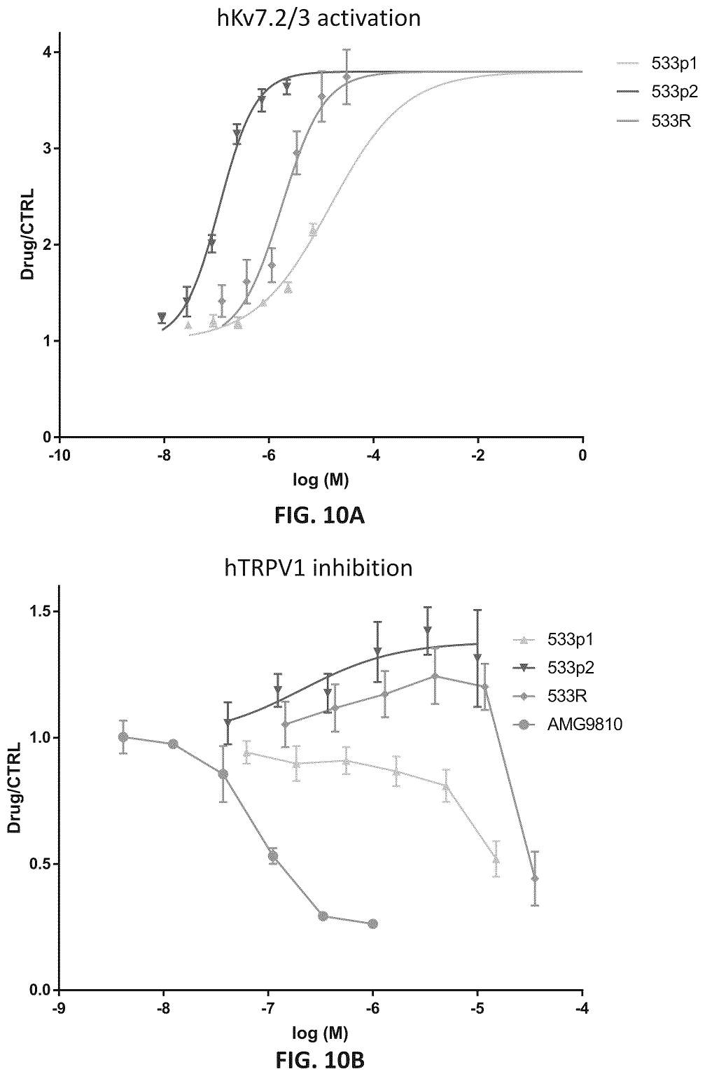

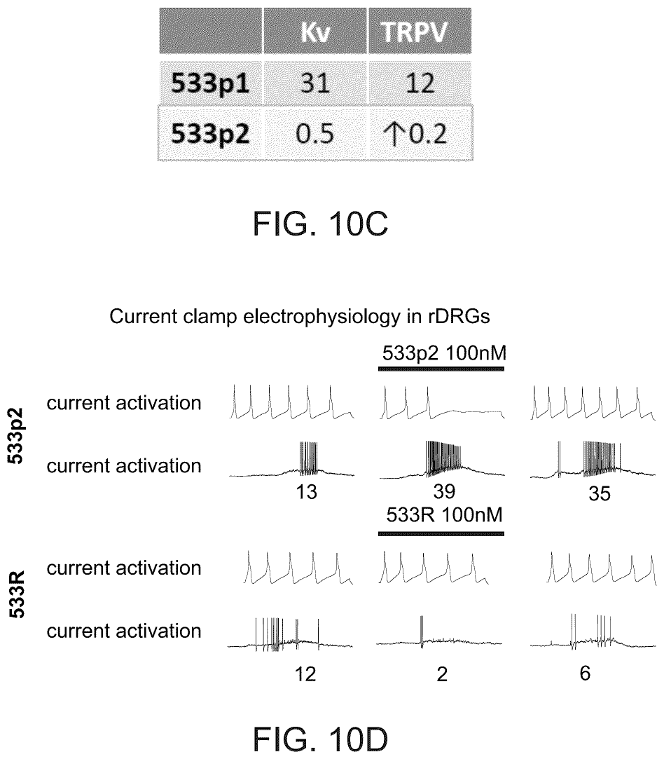

FIGS. 10A-C present the inhibitory potency of the tested compounds and reference compounds on current-induced and Capsaicin-induced neuronal activity, and show that a stereospecific modulation can be seen in 533 compound, such that 533P1 shows primarily activation of Kv7.2/3 and TRPV1 inhibition while 533P2 shows a more potent activation of Kv7.2/3 while activating TRPV1. This is exemplified in FIG. 10D, by current and capsaicin induced AP in rat DRG, in which 533P2 inhibits current induced AP and potentiates capsaicin induced AP while the racemic mixture which combines the effects of the two enantiomers shows reduced current induced AP inhibition while highly potentiating capsaicin induced AP inhibition.

### Dual Cross-Talk Modulation:

Compound 627 (see, FIG. 1 and Table 1) was selected for gaining further insight on the mechanism of action.

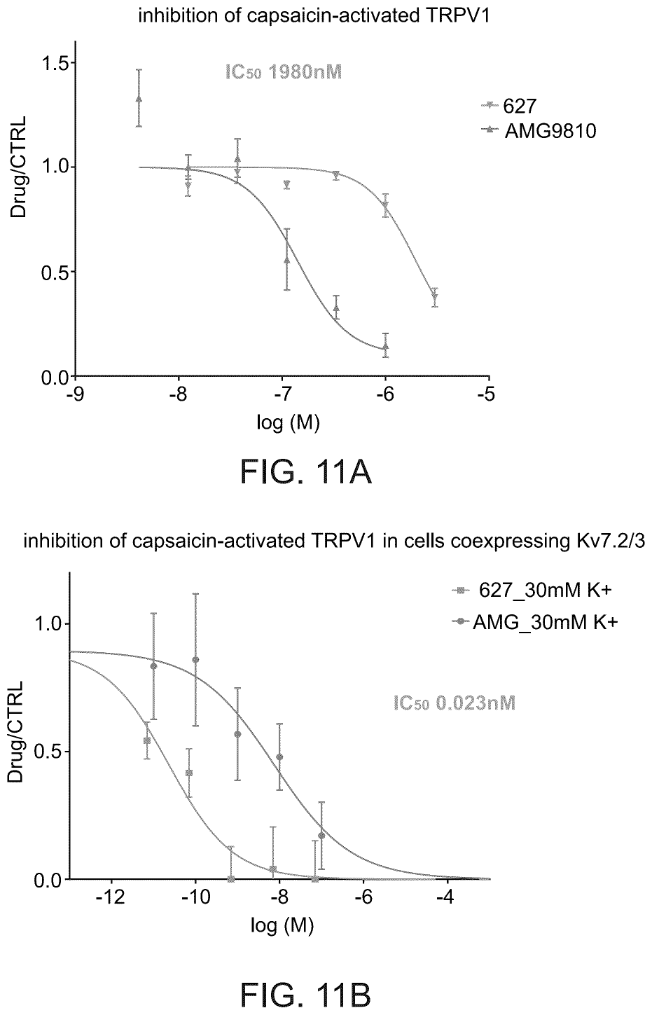

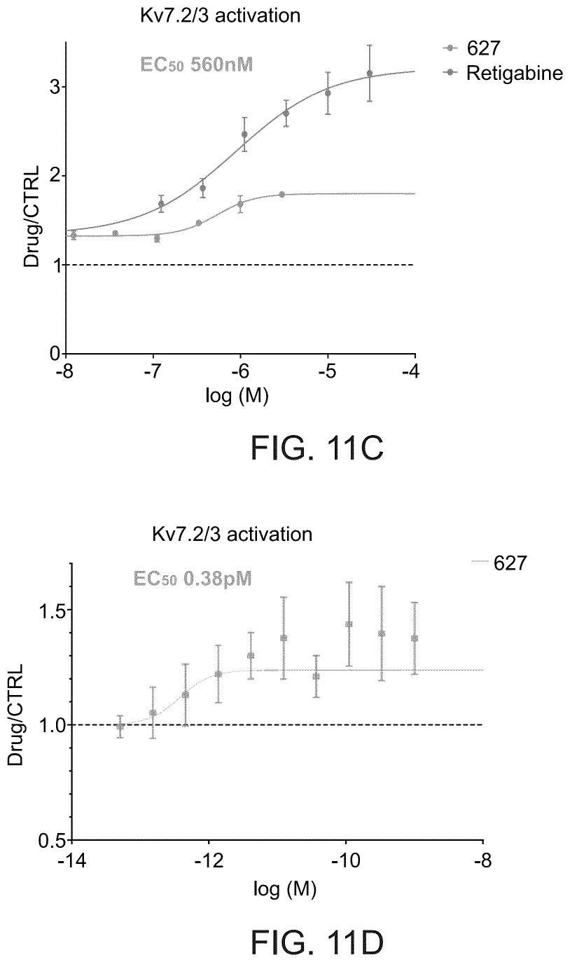

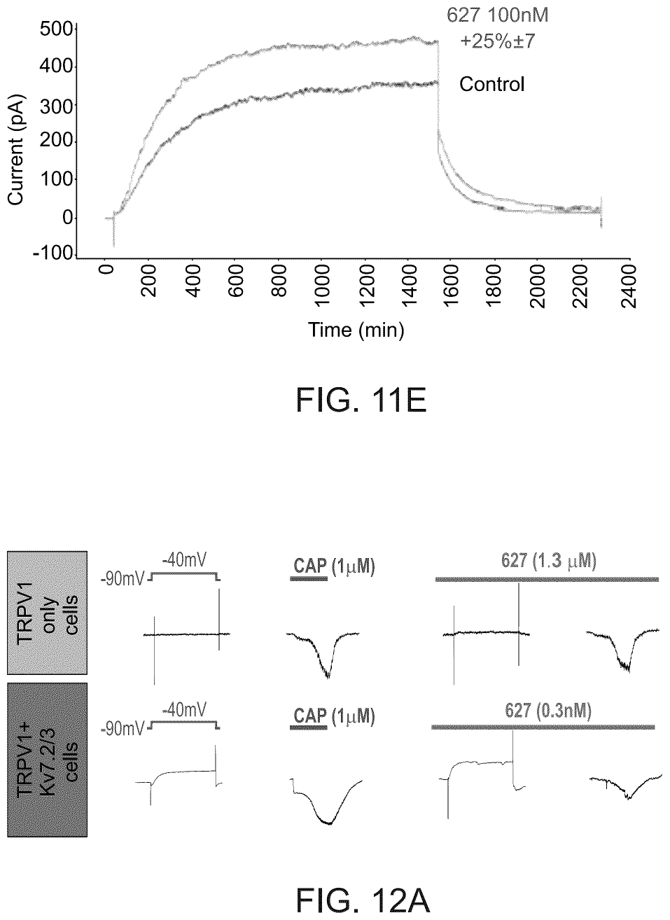

CHO cells expressing hTRPV1 or co-expressing hTRPV1 and hKv7.2/3 were treated with compound 627 (see. FIG. 1), with AMG9810, a known TRPV1 inhibitor used as a positive control, or with retigabine. The obtained data is presented in FIGS. 11A-E.

FIGS. 11A-B present comparative plots showing the TRPV1 inhibition by compound 627 compared to retigabine. It can be seen that when hKv7.2/3 is co-expressed and activated using chemical depolarization, the potency of compound 627 increases significantly, shifting leftward the dose-response curve, significantly stronger than the inhibition potency of AMG9810.

FIGS. 11C-D present plots demonstrating the inhibition of cells co-expressing hKv7.2/3, and showing that compound 627 at low concentration displays a significant hKv7.2/3 activation even at picomolar concentrations (FIG. 11D).

These data show that compound 627 displays a TRPV1 IC₅₀ of 1.9 μM and Kv7.2/3 EC₅₀ of 0.5 μM, when applied to CHO cell-lines solely expressing hKv7.2/3 or hTRPV1, as measured using fluorescent HCS, and displays about 30% Kv7.2/3 activation already at a concentration as low as 10 nM range.

As can be seen in FIG. 11E, activation of hKv7.2/3 activity by compound 627 was confirmed using electrophysiology, measuring whole-cell currents of hKv7.2/3 expressed in CHO cells, clamping membrane potential to −40 mV (1.5 s) (from a holding potential of −90 mV) (N=4). Compound 627 at 100 nM showed Kv7.2/3 current activation by 24.4±10.4%.

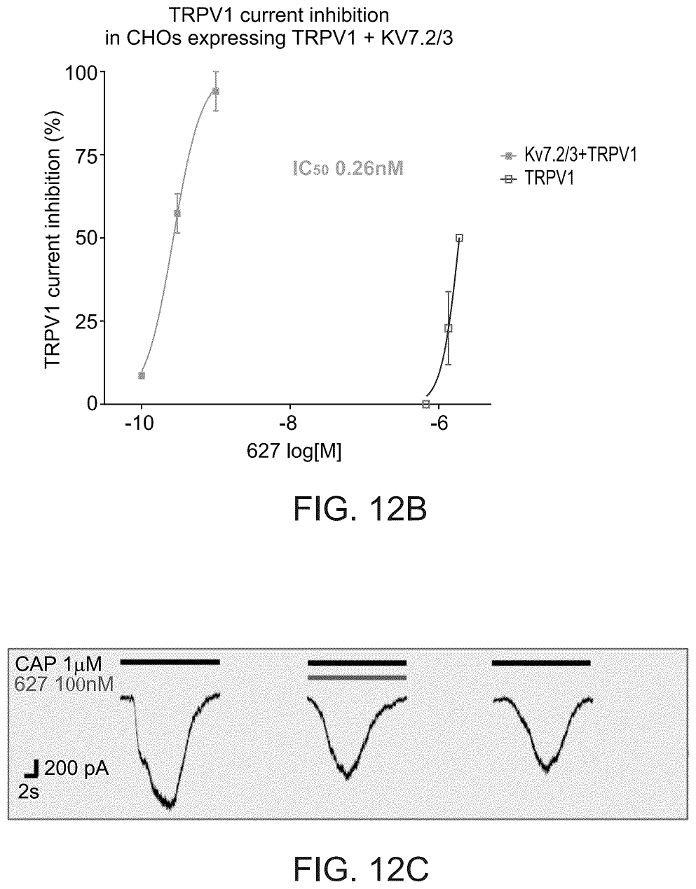

To exemplify the functional coupling of Kv7.2/3 and TRPV1, stably expressing Kv7.2/3 cells were transfected with human TRPV1 and their response to current ramp to −40 mV (Kv activation) and capsaicin (TRPV1 activation) was examined electrophysiologically. The obtained data is shown in FIGS. 12A-C.

FIG. 12A, upper panel, shows the effect of hKv7.2/3 co-expression in gaining potent 627 hTRPV1 inhibition. While hTRPV1 expressing cells show partial TRPV1 inhibition by 1.3 μM of compound 627, when hKv7.2/3 is co-expressed and activated (lower panel), compound 627 gains subnanomolar hTRPV1 inhibition potency.

FIG. 12B presents comparative dose-response plots displaying the hTRPV1 inhibition-potency gained (leftward shift) in the presence of compound 627 when hKv7.2/3 is co-expressed and activated.

FIG. 12C presents exemplary currents response to capsaicin in CHO cells co-expressing hTRPV1 and hKv7.2/3, but without hKv7.2/3 activation, in the presence of compound 627, and show that it displays a lack of hTRPV1 inhibition-potency gain, even at a concentration of 100 nM.

The data presented in FIGS. 12A-C indicate that the potentiation of the TRPV1 inhibitory potency increases by 4 orders of magnitude (from 2 μM to 0.26 nM) and requires Kv7.2/3. In addition, this potentiation of TRPV1 inhibition has a precondition of Kv7.2/3 activation as it is shown that without precondition of current ramp to −40 mV (Kv activation) TRPV1 could not be inhibited even at 100 nM.

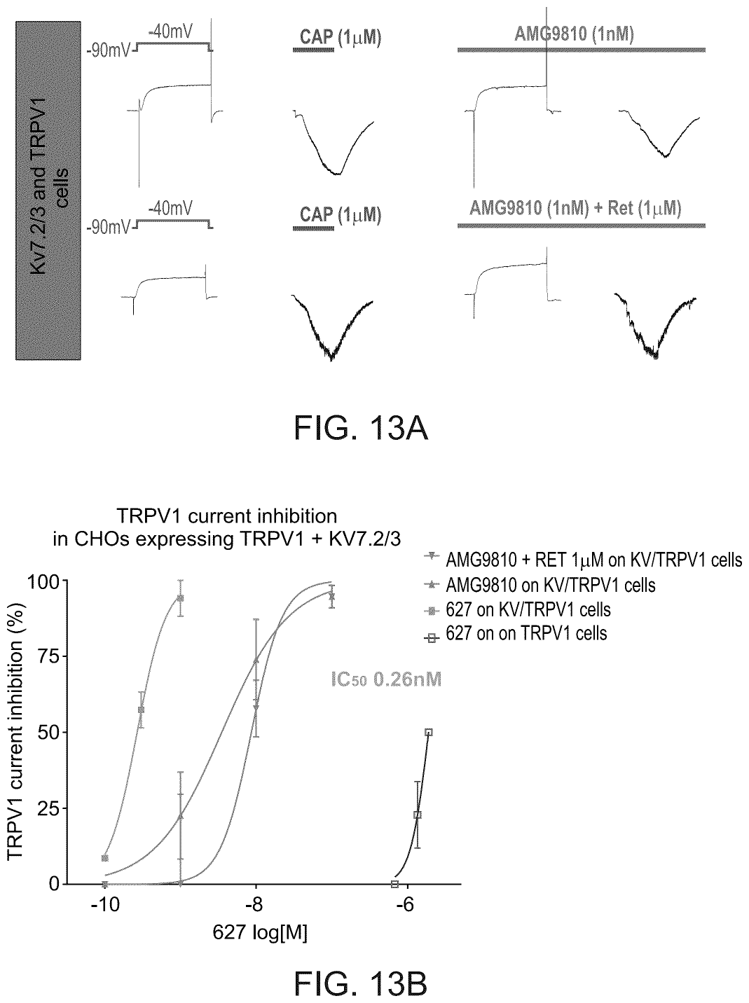

FIG. 13A shows that in the presence of AMG9810, a known TRPV1-inhibitor, failure to gain TRPV1-inhibition potency is observed, even when it is co-applied with the known Kv7.2/3 opener retigabine, in cells co-expressing both hKv7.2/3 and hTRPV1.

FIG. 13B presents comparative dose-response plots demonstrating similar hTRPV1 inhibition-potency of AMG9810 and of AMG9810 and retigabine combination treatment, indicating no synergistic effect therebetween. Contrary, compound 627 exhibits different hTRPV1 inhibition-potency when hKv7.2/3 is co-expressed and activated or when hTRPV1 is expressed alone. These data show that TRPV1 inhibition is uniquely effected by the same compound when Kv7.2/3 is co-expressed and activated.

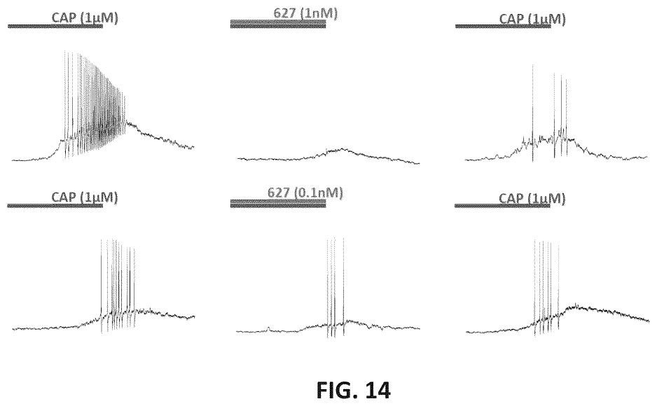

FIG. 14 presents an exemplary rat DRG membrane-potential recording showing action-potentials trains in response to Capsaicin application in the presence of compound 627. As can be seen, application of compound 627 at a concentration of 1 nM completely blocked the Capsaicin-evoked firring (upper panel), and about 50% inhibition of action-potentials firing was observed when compound 627 was applied at a concentration of 0.1 nM (0.04 ng/ml).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A compound represented by Formula III: 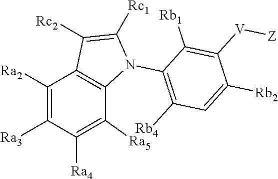
Formula III
wherein:
D is N or C—Rb3;
V is —(CR₂R₃)k-U;
k is 0, 1 or 2;
R₂ and R₃ are each independently hydrogen, alkyl, cycloalkyl and aryl;
U is amide (—C(═O)—NR₁₀—) or an isostere thereof;
Z is represented by Formula II: 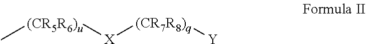
wherein:
u and q are each independently an integer of from 0 to 4, provided that u+q is at least 2;
X is selected from —O— and —NR₉—, or is absent;
Y is selected from OR₁₁, SR₁₁, NR₁₂R₁₃ and —NR₁₂—C(═O)—R₁₄;
R₅, R₆, R₇ and R₈ are each independently selected from hydrogen, halo, alkyl, haloalkyl, cycloalkyl, heteroalicyclic, aryl, alkylamine, alkoxy, haloalkoxy, hydroxy, ether and aryloxy, or, alternatively, two of R₅, R₆, R₇, R₈ and R₉ form together an alicyclic or heteroalicyclic ring;
R₉, R₁₀, R₁₁, R₁₂, R₁₃ and R₁₄ are each independently selected from hydrogen, alkyl, cycloalkyl, and aryl, or, alternatively, two of R₅, R₆, R₇, R₈, R₉ and R₁₁ or two of R₅, R₆, R₇, R₈, R₉, R₁₂ and R₁₃ form together an alicyclic or heteroalicyclic ring; and
Ra2-Ra5, Rb1-Rb4, Rc1 and Rc2 are each independently a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide,
and wherein:
when D is C—Rb3, Rd l is hydrogen and Rc2 is selected from halo, alkyl and haloalkyl.

2. The compound of claim 1, wherein D is N.

3. The compound of claim 1, wherein at least one of Rb1-Rb4 is halo.

4. The compound of claim 1, wherein at least two of Ra2-Ra5 are each independently selected from halo and alkoxy.

5. The compound of claim 1, wherein Ra3 and Ra5 are each independently a halo.

6. The compound of claim 1, wherein X is absent.

7. The compound of claim 1, wherein at least one of R₅, R₆, R₇ and R₈ is independently selected from alkyl, hydroxy, alkoxy, haloalkyl, ether, and halo, and/or at least two of R₅, R₆, R₇ and R₈ form together an alicyclic or heteroalicyclic ring.

8. The compound of claim 1, wherein Y is OR₁₁, and R₁₁ is hydrogen or an alkyl.

9. A compound represented by Formula I: 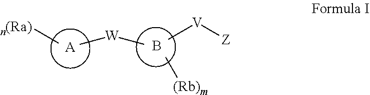
wherein:
A and B are each independently selected from an aryl and a heteroaryl;
W is selected from —S—, —O—, —CR₁(OH)—, —C(═O), and NRx, wherein said Rx forms a nitrogen-containing heterocyclic ring with one of the Ra substituents of ring A;
n is an integer of from 1 to 5;
m is an integer of from 0 to 4;
Ra and Rb are each independently a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Ra substituents form together an alicyclic or heterocyclic ring, at least one of the Ra substituents forms together with Rx, if present, said nitrogen-containing heterocyclic ring and/or at least two of the Rb substituents form together an alicyclic or heterocyclic ring, wherein when n is greater than 1, each Ra is the same or different substituent, and when m is greater than 1, each Rb is the same or different substituent;
R₁, if present, is hydrogen or alkyl;
V is —(CR₂R₃)k-U;
k is 0, 1 or 2;
R₂ and R₃ are each independently hydrogen, alkyl, cycloalkyl and aryl;
U is amide (—C(═O)—NR₁₀—) or an isostere thereof; and
Z is represented by Formula II: 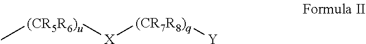
wherein:
u and q are each independently an integer of from 0 to 4, provided that u+q is at least 2;
X is selected from —O— and —NR₉—, or is absent;
Y is OR₁₁;
R₅, R₆, R₇ and R₈ are each independently selected from hydrogen, halo, alkyl, haloalkyl, cycloalkyl, heteroalicyclic, aryl, alkylamine, alkoxy, haloalkoxy, hydroxy, ether and aryloxy, or, alternatively, two of R₅, R₆, R₇, R₈ and R₉ form together an alicyclic or heteroalicyclic ring; and
R₉, R₁₀, R₁₁, R₁₂, R₁₃ and R₁₄ are each independently selected from hydrogen, alkyl, cycloalkyl, and aryl, or, alternatively, two of R₅, R₆, R₇, R₈, R₉ and R₁₁ or two of R₅, R₆, R₇, R₈, R₉, R₁₂ and R₁₃ form together an alicyclic or heteroalicyclic ring,
and wherein when W is —S—, —O—, —CR₁(OH)—, or —C(═O), at least one of said Ra substituent(s) is selected from alkyl, haloalkyl, cycloalkyl and aryl and is at the ortho position with respect to said W; and
when said Rx forms said nitrogen-containing heterocyclic ring with one of the Ra substituents of ring A, ring B is a heteroaryl.

10. The compound of claim 9, wherein m is other than 0 and at least one of said Rb substituent(s) is halo.

11. The compound of claim 9, wherein n is 3, 4 or 5.

12. The compound of claim 9, being represented by Formula Ia or Ib: 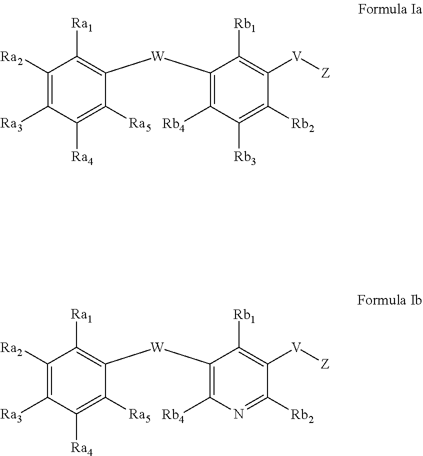
wherein:
W, V and Z are as defined for Formula I;
when W is —S—, —O—, —CR₁(OH)—, or —C(═O), Ra1 is selected from alkyl, haloalkyl, cycloalkyl and aryl;
when said Rx forms said nitrogen-containing heterocyclic ring with one of the Ra substituents of ring A, the compound has said Formula Ib; and
Ra2-Ra5, Rb1, Rb2, Rb4 and Rb3, if present, are each independently a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide.

13. The compound of claim 9, wherein X is absent.

14. The compound of claim 13, wherein at least one of R₅, R₆, R₇ and R₈ is independently selected from alkyl, hydroxy, alkoxy, haloalkyl, ether, and halo, and/or at least two of R₅, R₆, R₇ and R₈ form together an alicyclic or heteroalicyclic ring.

15. A pharmaceutical composition comprising the compound of claim 1 and a pharmaceutically acceptable carrier.

16. A pharmaceutical composition comprising the compound of claim 9 and a pharmaceutically acceptable carrier.

17. A method of modulating an activity of a voltage-dependent potassium channel and/or for modulating an activity of TRPV1 channel, the method comprising contacting a cell, a tissue or an organ which express the channel with the compound of claim 1.

18. A method of treating a medical condition associated with an activity of a voltage-dependent potassium channel and/or a TRPV1 channel in a subject in need thereof, the method comprising administering to the subject the compound of claim 1.

19. The method of claim 18, wherein said medical condition is neuropathic pain, pruritus or tinnitus.

20. A method of modulating an activity of a voltage-dependent potassium channel and/or for modulating an activity of TRPV1 channel, the method comprising contacting a cell, a tissue or an organ which express the channel with the compound of claim 9.

21. A method of treating a medical condition associated with an activity of a voltage-dependent potassium channel and/or a TRPV1 channel in a subject in need thereof, the method comprising administering to the subject the compound of claim 9.

22. The method of claim 21, wherein said medical condition is neuropathic pain, pruritus or tinnitus.
